(19) United States
(12) Patent Application Publication
(10) Pub. No.: US 2008/0032305 A1
(43) Pub. Date: Feb. 7, 2008

(54) Methods and compositions for analysis of UGT1A1 alleles

(75) Inventors: Erin Dorn, Cross Plains, WI (US); Eric B. Rasmussen, San Diego, CA (US)

Correspondence Address: MEDLEN & CARROLL, LLP, 101 HOWARD STREET, SUITE 350, SAN FRANCISCO, CA, 94105, US

(73) Assignee: Third Wave Technologies, Inc., Madison, WI (US)

(21) Appl. No.: 11/799,765

(22) Filed: May 1, 2007

Related U.S. Application Data
(63) Continuation-in-part of application No. 10/354,953, filed on Jan. 30, 2003, now abandoned.
(60) Provisional application No. 60/353,444, filed on Jan. 31, 2002.
(60) Provisional application No. 60/372,475, filed on Apr. 15, 2002.
(60) Provisional application No. 60/366,984, filed on Mar. 22, 2002.
(60) Provisional application No. 60/356,326, filed on Feb. 13, 2002.

(51) Int. Cl. C12Q 1/68

(52) U.S. Cl. 435/6

(57) ABSTRACT

The present invention relates to methods for detecting polymorphisms in enzymes related to drug metabolizm (Drug Metabolizing Enzymes or DMEs) such as uridine diphosphate glucuronosyl transferase (UGT) gene promoter, with nucleic acid detection assays. The present invention also relates to detection assay kits.

The present application is a continuation-in-part of U.S. patent application Ser. No. 10/354,953, which claims priority to U.S. Provisional Applications 60/353,444, filed Jan. 31, 2002, 60/372,475, filed Apr. 15, 2002, 60/366,984, filed Mar. 22, 2002, and 60/356,326, filed Feb. 13, 2002, each of which is herein incorporated by reference.

### FIELD OF THE INVENTION

The present invention relates to methods for detecting polymorphisms in enzymes related to drug metabolizm (Drug Metabolizing Enzymes or DMEs) such as uridine diphosphate glucuronosyl transferase (UGT) gene promoter, cytochrome p450, with a non-amplified oligonucleotide detection assays. The present invention also relates to pharmacogenetic DME detection assay kits.

### BACKGROUND

As the Human Genome Project nears completion and the volume of genetic sequence information available increases, genomics research and subsequent drug design efforts increase as well. There exists a need for systems and methods that allow for the efficient ordering, development, production and sales of detection assays that can be used in genomics research, drug design, and personalized medicine. A number of institutions are actively mining the available genetic sequence information to identify correlations between genes, gene expression and phenotypes (e.g., disease states, metabolic responses, and the like). These analyses include an attempt to characterize the effect of gene mutations and genetic and gene expression heterogeneity in individuals and populations. However, despite the wealth of sequence information available, information on the frequency and clinical relevance of many polymorphisms and other variations has yet to be obtained and validated. For example, the human reference sequences used in current genome sequencing efforts do not represent an exact match for any one person's genome. In the Human Genome Project (HGP), researchers collected blood (female) or sperm (male) samples from a large number of donors. However, only a few samples were processed as DNA resources, and the source names are protected so neither donors nor scientists know whose DNA is being sequenced. The human genome sequence generated by the private genomics company Celera was based on DNA samples collected from five donors who identified themselves as Hispanic, Asian, Caucasian, or African-American. The small number of human samples used to generate the reference sequences does not reflect the genetic diversity among population groups and individuals. Attempts to analyze individuals based on the genome sequence information will often fail. For example, many genetic detection assays are based on the hybridization of probe oligonucleotides to a target region on genomic DNA or mRNA. Probes generated based on the reference sequences will often fail (e.g., fail to hybridize properly, fail to properly characterize the sequence at specific position of the target) because the target sequence for many individuals differs from the reference sequence. Differences may be on an individual-by-individual basis, but many follow regional population patterns (e.g., many correlate highly to race, ethnicity, geographic local, age, environmental exposure, etc.). With the limited utility of information currently available, the art is in need of systems and methods that can optionally be used in one or more production facilities for acquiring, analyzing, storing, and applying large volumes of genetic information with the goal of providing an array of one or more types of detection assay technologies for research and clinical analysis of biological samples. It is an object of the invention to fill these various needs.

### SUMMARY OF THE INVENTION

The present invention relates to methods for detecting polymorphisms in a uridine diphosphate glucuronosyl transferase (UGT) gene promoter with detection assays. The present invention also relates to pharmacogenetic UGT detection assay kits.

In some embodiments, the present invention provides a composition comprising an detection assay configured for detecting at least one polymorphism in UGT1A1. In some embodiments, said oligonucleotide detection assay comprises a non-amplified detection assay. In certain embodiments, the at least one polymorphism in UGT1A1 is selected from UGT1A1 *6, *27, *28, and the polymorphisms shown in FIG. 4.

In some preferred embodiments, the detection assay comprises a primary probe, an INVADER oligonucleotide, a structure specific enzyme, and a FRET cassette. In particularly preferred embodiments, the primary probe comprises a 5′ flap. In some particularly preferred embodiments, the a detection assay of the present invention comprises at least one sequence shown in FIG. 1, 2, 3, 5, 6, 7, 10, 11, 12, 13, 14, 15, 21, or 22. In particularly preferred embodiments, the assay comprises an oligonucleotide that consists of a first portion having a sequence selected from the group consisting of SEQ ID NOS: 964, 967, 970 and 973, and a second portion consisting of a 5′ arm.

In some embodiments, the present invention provides a method comprising;
a) providing:
   i) a composition comprising an oligonucleotide detection assay configured for detecting a UGT1A1 polymorphism, and
   ii) a sample from a subject; and
b) testing said sample with said composition in order to determine if said subject has said UGT1A1 polymorphism.

In some embodiments, the UGT1A1 polymorphism is selected from UGT1A1 *6, *27, *28, and the polymorphisms shown in FIG. 4.

In some embodiments of the methods of the present invention, the oligonucleotide detection assay is a non-amplified oligonucleotide detection assay. In certain preferred embodiments, the oligonucleotide detection assay comprises a primary probe, an INVADER oligonucleotide, a structure specific enzyme, and a FRET cassette. In some particularly preferred embodiments, the primary probe comprises a 5′ flap.

In some preferred embodiments, the a detection assay of the present invention comprises an oligonucleotide at least one sequence shown in FIG. 1, 2, 3, 5, 6, 7, 10, 11, 12, 13, 14, 15, 21, or 22. In particularly preferred embodiments, the assay comprises an oligonucleotide that consists of a first portion having a sequence selected from the group consisting of SEQ ID NOS: 964, 967, 970 and 973, and a second portion consisting of a 5′ arm.

In some embodiments, the present invention provides methods for detecting TA5 and TA8 UGT repeats in a sample comprising, contacting a sample comprising a target sequence with an oligonucleotide detection assay, and determining if the target contains UGT TA5 and/or TA8 repeats. In some embodiments, the detection assay is a non-amplified oligonucleotide detection assay. In some preferred embodiments, the assay comprises an INVADER assay.

The present invention provides systems, methods, and kits employing nucleic acid detection assays to screen subjects in order to facilitate drug therapy and avoid problems of toxicity or lack of efficacy. In particular, the present invention provides systems, methods, and kits with a nucleic acid detection assay configured to detect polymorphisms in gene sequences associated with Irinotecan safety or efficacy. In this regard, the present invention allows the identification of subjects as suitable or not suitable for treatment with Irinotecan based on the results of employing the detection assay on a sample from the subject.

### DESCRIPTION OF THE FIGURES

The following figures form part of the present specification and are included to further demonstrate certain aspects and embodiments of the present invention. The invention may be better understood by reference to one or more of these figures in combination with the description of specific embodiments presented herein.

FIG. 1 shows oligonucleotides for an exemplary INVADER assay for detecting UGT1A1*6.

FIG. 2 shows oligonucleotides for an exemplary INVADER assay for detecting UGT1A1*27.

FIG. 3 shows oligonucleotides for an exemplary INVADER assay for detecting UGT1A1*28.

FIG. 4 shows set of nine polymorphisms in human UGT1A1.

FIG. 5 shows exemplary detection assays (INVADER assays) for the nine UGT1A1 polymorphisms shown in FIG. 4.

FIG. 6 shows exemplary detection probes for detection of UGT1A1*28 alleles. “Hex” indicates a hexanediol 3′ blocking group.

FIG. 7 shows exemplary detection probes for detection of UGT1A1*28 alleles. “Hex” indicates a hexanediol 3′ blocking group.

FIG. 8 shows an Excel graph showing detection of UGT1A1*28 wild-type (WT), insertion (Ins) alleles in samples of genomic DNA.

FIG. 9 shows Excel graphs of detection of UGT1A1*28 WT and Ins alleles in reactions having different amounts of target DNA.

FIG. 10 shows exemplary INVADER assay configurations for TA5, TA6, TA7, and TA8 UGT1A1*28 detection.

FIG. 11 shows an exemplary INVADER assay configuration for TA5 UGT1A1*28 detection.

FIG. 12 shows an exemplary INVADER assay configuration for TA6 UGT1A1*28 detection.

FIG. 13 shows an exemplary INVADER assay configuration for TA7 UGT1A1*28 detection.

FIG. 14 shows an exemplary INVADER assay configuration for TA8 UGT1A1*28 detection.

FIG. 15 shows an exemplary INVADER assay design for an internal control (Alpha Actin) that may be used with UGT detection assays.

FIG. 16 shows certain results of the UGT Example 2.

FIG. 17 shows certain results of the UGT Example 2.

FIG. 18 shows certain colon cancer management protocols.

FIG. 19 shows certain colon cancer management protocols.

FIG. 20 shows certain colon cancer management protocols.

FIG. 21 shows oligonucleotides for an exemplary INVADER assay for detecting UGT1A1*27.

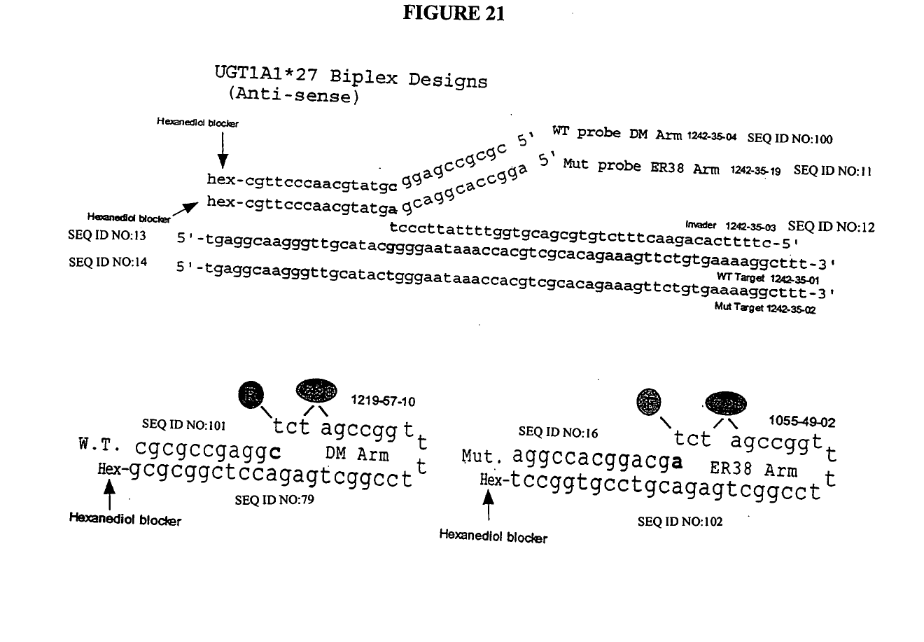

FIG. 22 shows oligonucleotides for an exemplary INVADER assay for detecting UGT1A1 *28.

### DEFINITIONS

To facilitate an understanding of the present invention, a number of terms and phrases are defined below:

As used herein, the terms “solid support” or “support” refer to any material that provides a solid or semi-solid structure with which another material can be attached. Such materials include smooth supports (e.g., metal, glass, plastic, silicon, and ceramic surfaces) as well as textured and porous materials. Such materials also include, but are not limited to, gels, rubbers, polymers, and other non-rigid materials. Solid supports need not be flat. Supports include any type of shape including spherical shapes (e.g., beads). Materials attached to solid support may be attached to any portion of the solid support (e.g., may be attached to an interior portion of a porous solid support material). Preferred embodiments of the present invention have biological molecules such as nucleic acid molecules and proteins attached to solid supports. A biological material is “attached” to a solid support when it is associated with the solid support through a non-random chemical or physical interaction. In some preferred embodiments, the attachment is through a covalent bond. However, attachments need not be covalent or permanent. In some embodiments, materials are attached to a solid support through a “spacer molecule” or “linker group.” Such spacer molecules are molecules that have a first portion that attaches to the biological material and a second portion that attaches to the solid support. Thus, when attached to the solid support, the spacer molecule separates the solid support and the biological materials, but is attached to both.

As used herein, the term “derived from a different subject,” such as samples or nucleic acids derived from a different subjects refers to a samples derived from multiple different individuals. For example, a blood sample comprising genomic DNA from a first person and a blood sample comprising genomic DNA from a second person are considered blood samples and genomic DNA samples that are derived from different subjects. A sample comprising five target nucleic acids derived from different subjects is a sample that includes at least five samples from five different individuals. However, the sample may further contain multiple samples from a given individual.

As used herein, the term “treating together,” when used in reference to experiments or assays, refers to conducting experiments concurrently or sequentially, wherein the results of the experiments are produced, collected, or analyzed together (i.e., during the same time period). For example, a plurality of different target sequences located in separate wells of a multiwell plate or in different portions of a microarray are treated together in a detection assay where detection reactions are carried out on the samples simultaneously or sequentially and where the data collected from the assays is analyzed together.

The terms “assay data” and “test result data” as used herein refer to data collected from performance of an assay (e.g., to detect or quantitate a gene, SNP or an RNA). Test result data may be in any form, i.e., it may be raw assay data or analyzed assay data (e.g., previously analyzed by a different process). Collected data that has not been further processed or analyzed is referred to herein as “raw” assay data (e.g., a number corresponding to a measurement of signal, such as a fluorescence signal from a spot on a chip or a reaction vessel, or a number corresponding to measurement of a peak, such as peak height or area, as from, for example, a mass spectrometer, HPLC or capillary separation device), while assay data that has been processed through a further step or analysis (e.g., normalized, compared, or otherwise processed by a calculation) is referred to as “analyzed assay data” or “output assay data”.

As used herein, the term “database” refers to collections of information (e.g., data) arranged for ease of retrieval, for example, stored in a computer memory. A “genomic information database” is a database comprising genomic information, including, but not limited to, polymorphism information (i.e., information pertaining to genetic polymorphisms), genome information (i.e., genomic information), linkage information (i.e., information pertaining to the physical location of a nucleic acid sequence with respect to another nucleic acid sequence, e.g., in a chromosome), and disease association information (i.e., information correlating the presence of or susceptibility to a disease to a physical trait of a subject, e.g., an allele of a subject). “Database information” refers to information to be sent to databases, stored in a database, processed in a database, or retrieved from a database. “Sequence database information” refers to database information pertaining to nucleic acid sequences. As used herein, the term “distinct sequence databases” refers to two or more databases that contain different information than one another. For example, the dbSNP and GenBank databases are distinct sequence databases because each contains information not found in the other.

As used herein the term “set of oligonucleotides” means at least two oligonucleotides that differ in at least one characteristic (e.g., sequence, purity, required buffer, required salt concentration).

As used herein the term “purified sample,” as in a purified oligonucleotide sample, refers to a sample where the full-length oligonucleotide in a sample is the predominate species of oligonucleotide. For example, in some embodiments, at least 90%, preferably 95%, and more preferably 99% of oligonucleotides in a sample are full-length oligonucleotides.

As used herein, the terms “SNP,” “SNPs” or “single nucleotide polymorphisms” refer to single base changes at a specific location in an organism's (e.g., a human) genome. “SNPs” can be located in a portion of a genome that does not code for a gene. Alternatively, a “SNP” may be located in the coding region of a gene. In this case, the “SNP” may alter the structure and function of the RNA or the protein with which it is associated.

As used herein, the term “allele” refers to a variant form of a given sequence (e.g., including but not limited to, genes containing one or more SNPs). A large number of genes are present in multiple allelic forms in a population. A diploid organism carrying two different alleles of a gene is said to be heterozygous for that gene, whereas a homozygote carries two copies of the same allele.

As used herein, the term “linkage” refers to the proximity of two or more markers (e.g., genes) on a chromosome.

As used herein, the term “allele frequency” refers to the frequency of occurrence of a given allele (e.g., a sequence containing a SNP) in given population (e.g., a specific gender, race, or ethnic group). Certain populations may contain a given allele within a higher percent of its members than other populations. For example, a particular mutation in the breast cancer gene called BRCA1 was found to be present in one percent of the general Jewish population. In comparison, the percentage of people in the general U.S. population that have any mutation in BRCA1 has been estimated to be between 0.1 to 0.6 percent. Two additional mutations, one in the BRCA1 gene and one in another breast cancer gene called BRCA2, have a greater prevalence in the Ashkenazi Jewish population, bringing the overall risk for carrying one of these three mutations to 2.3 percent.

As used herein, the term “in silico analysis” refers to analysis performed using computer processors and computer memory. For example, “insilico SNP analysis” refers to the analysis of SNP data using computer processors and memory.

As used herein, the term “genotype” refers to the actual genetic make-up of an organism (e.g., in terms of the particular alleles carried at a genetic locus). Expression of the genotype gives rise to an organism's physical appearance and characteristics—the “phenotype.”

As used herein, the term “locus” refers to the position of a gene or any other characterized sequence on a chromosome.

As used herein the term “disease” or “disease state” refers to a deviation from the condition regarded as normal or average for members of a species, and which is detrimental to an affected individual under conditions that are not inimical to the majority of individuals of that species (e.g., diarrhea, nausea, fever, pain, and inflammation etc).

As used herein, the term “treatment” in reference to a medical course of action refer to steps or actions taken with respect to an affected individual as a consequence of a suspected, anticipated, or existing disease state, or wherein there is a risk or suspected risk of a disease state. Treatment may be provided in anticipation of or in response to a disease state or suspicion of a disease state, and may include, but is not limited to preventative, ameliorative, palliative or curative steps. The term “therapy” refers to a particular course of treatment.

The term “gene” refers to a nucleic acid (e.g., DNA) sequence that comprises coding sequences necessary for the production of a polypeptide, RNA (e.g., rRNA, tRNA, etc.), or precursor. The polypeptide, RNA, or precursor can be encoded by a full length coding sequence or by any portion of the coding sequence so long as the desired activity or functional properties (e.g., ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments included when a gene is transcribed into heterogeneous nuclear RNA (hnRNA); introns may contain regulatory elements such as enhancers. Introns are removed or “spliced out” from the nuclear or primary transcript; introns therefore are generally absent in the messenger RNA (mRNA) transcript. The mRNA functions during translation to specify the sequence or order of amino acids in a nascent polypeptide. Variations (e.g., mutations, SNPS, insertions, deletions) in transcribed portions of genes are reflected in, and can generally be detected in corresponding portions of the produced RNAs (e.g., hnRNAs, mRNAs, rRNAs, tRNAs).

Where the phrase “amino acid sequence” is recited herein to refer to an amino acid sequence of a naturally occurring protein molecule, amino acid sequence and like terms, such as polypeptide or protein are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified,” “mutant,” and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. In this case, the DNA sequence thus codes for the amino acid sequence.

DNA and RNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotides or polynucleotide, referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or, in other words, the nucleic acid sequence that encodes a gene product. The coding region may be present in either a cDNA, genomic DNA, or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the terms “complementary” or “complementarity” are used in reference to polynucleotides (i.e., a sequence of nucleotides) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is one that at least partially inhibits a completely complementary sequence from hybridizing to a target nucleic acid and is referred to using the functional term “substantially homologous.” The term “inhibition of binding,” when used in reference to nucleic acid binding, refers to inhibition of binding caused by competition of homologous sequences for binding to a target sequence. The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that lacks even a partial degree of complementarity (e.g., less than about 30% identity); in the absence of non-specific binding the probe will not hybridize to the second non-complementary target.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.).

When used in reference to a double-stranded nucleic acid sequence such as a cDNA or genomic clone, the term “substantially homologous” refers to any probe that can hybridize to either or both strands of the double-stranded nucleic acid sequence under conditions of low stringency as described above.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization [1985]). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50×Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

The following terms are used to describe the sequence relationships between two or more polynucleotides: “reference sequence,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length cDNA sequence given in a sequence listing or may comprise a complete gene sequence. Generally, a reference sequence is at least 20 nucleotides in length, frequently at least 25 nucleotides in length, and often at least 50 nucleotides in length. Since two polynucleotides may each (1) comprise a sequence (i.e., a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e., gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman [Smith and Waterman, Adv. Appl. Math. 2: 482 (1981)] by the homology alignment algorithm of Needleman and Wunsch [Needleman and Wunsch, J. Mol. Biol. 48:443 (1970)], by the search for similarity method of Pearson and Lipman [Pearson and Lipman, Proc. Natl. Acad. Sci. (U.S.A.) 85:2444 (1988)], by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected. The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

As applied to polynucleotides, the term “substantial identity” denotes a characteristic of a polynucleotide sequence, wherein the polynucleotide comprises a sequence that has at least 85 percent sequence identity, preferably at least 90 to 95 percent sequence identity, more usually at least 99 percent sequence identity as compared to a reference sequence over a comparison window of at least 20 nucleotide positions, frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison. The reference sequence may be a subset of a larger sequence, for example, as a splice variant of the full-length sequences.

As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions that are not identical differ by conservative amino acid substitutions. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q replicase, MDV-1 RNA is the specific template for the replicase (D. L. Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (D. Y. Wu and R. B. Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “amplifiable nucleic acid” is used in reference to nucleic acids that may be amplified by any amplification method. It is contemplated that “amplifiable nucleic acid” will usually comprise “sample template.” As used herein, the term “sample template” refers to nucleic acid originating from a sample that is analyzed for the presence of “target” (defined below). In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” or “hybridization probe” refers to an oligonucleotide (i.e., a sequence of nucleotides), whether occurring naturally as in a purified restriction digest or produced synthetically, recombinantly or by PCR amplification, that is capable of hybridizing, at least in part, to another oligonucleotide of interest. A probe may be single-stranded or double-stranded. Probes are useful in the detection, identification and isolation of particular sequences. In some preferred embodiments, probes used in the present invention will be labeled with a “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target” refers to a nucleic acid sequence or structure to be detected or characterized.

As used herein, the term “polymerase chain reaction” (“PCR”) refers to the method of K. B. Mullis (See e.g., U.S. Pat. Nos. 4,683,195, 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.” With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process itself are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “recombinant DNA molecule” as used herein refers to a DNA molecule that is comprised of segments of DNA joined together by means of molecular biological techniques.

As used herein, the term “antisense” is used in reference to RNA sequences that are complementary to a specific RNA sequence (e.g., mRNA). The term “antisense strand” is used in reference to a nucleic acid strand that is complementary to the “sense” strand. The designation (−) (i.e., “negative”) is sometimes used in reference to the antisense strand, with the designation (+) sometimes used in reference to the sense (i.e., “positive”) strand.

The term “isolated” when used in relation to a nucleic acid, as in “an isolated oligonucleotide” or “isolated polynucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids are nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acids encoding a polypeptide include, by way of example, such nucleic acid in cells ordinarily expressing the polypeptide where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may single-stranded), but may contain both the sense and anti-sense strands (i.e., the oligonucleotide or polynucleotide may be double-stranded).

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (e.g., 10 nucleotides, 11, . . . , 20, . . . ).

As used herein, the term “purified” or “to purify” refers to the removal of contaminants from a sample. As used herein, the term “purified” refers to molecules (e.g., nucleic or amino acid sequences) that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (J. Sambrook et al., Molecular Cloning: A Laboratory Manual, Cold Spring Harbor Press, NY, pp 9.31-9.58 [1989]).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of labeled antibodies.

The term “test compound” refers to any chemical entity, pharmaceutical, drug, and the like that are tested in an assay (e.g., a drug screening assay) for any desired activity (e.g., including but not limited to, the ability to treat or prevent a disease, illness, sickness, or disorder of bodily function, or otherwise alter the physiological or cellular status of a sample). Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; and fluorescent dyes alone or in combination with moieties that can suppress or shift emission spectra by fluorescence resonance energy transfer (FRET). Labels may provide signals detectable by fluorescence, radioactivity, colorimetry, gravimetry, X-ray diffraction or absorption, magnetism, enzymatic activity, and the like. A label may be a charged moiety (positive or negative charge) or alternatively, may be charge neutral. Labels can include or consist of nucleic acid or protein sequence, so long as the sequence comprising the label is detectable.

The term “signal” as used herein refers to any detectable effect, such as would be caused or provided by a label or an assay reaction.

The term “detection” as used herein refers to quantitatively or qualitatively identifying an analyte (e.g., DNA, RNA or a protein) within a sample. The term “detection assay” as used herein refers to a kit, test, or procedure performed for the purpose of detecting an analyte nucleic acid within a sample. Detection assays produce a detectable signal or effect when performed in the presence of the target analyte, and include but are not limited to assays incorporating the processes of hybridization, nucleic acid cleavage (e.g., exo- or endonuclease), nucleic acid amplification, nucleotide sequencing, primer extension, or nucleic acid ligation.

As used herein, the term “functional detection oligonucleotide” refers to an oligonucleotide that is used as a component of a detection assay, wherein the detection assay is capable of successfully detecting (i.e., producing a detectable signal) an intended target nucleic acid when the functional detection oligonucleotide provides the oligonucleotide component of the detection assay. This is in contrast to non-functional detection oligonucleotides, which fail to produce a detectable signal in a detection assay for the particular target nucleic acid when the non-functional detection oligonucleotide is provided as the oligonucleotide component of the detection assay. Determining if an oligonucleotide is a functional oligonucleotide can be carried out experimentally by testing the oligonucleotide in the presence of the particular target nucleic acid using the detection assay.

As used herein, the term “a detection assay configured for target detection” refers to a collection of assay components that are capable of producing a detectable signal when carried out using the target nucleic acid. For example, a detection assay that has empirically been demonstrated to detect a particular single nucleotide polymorphism is considered a detection assay configured for target detection.

As used herein, the phrase “unique detection assay” refers to a detection assay that has a different collection of detection assay components in relation to other detection assays located on the same detection panel. A unique assay doesn't necessarily detect a different target (e.g. SNP) than other assays on the same detection panel, but it does have a least one difference in the collection of components used to detect a given target (e.g. a unique detection assay may employ a probe sequences that is shorter or longer in length than other assays on the same detection panel).

As used herein, the term “candidate” refers to an assay or analyte, e.g., a nucleic acid, suspected of having a particular feature or property. A “candidate sequence” refers to a nucleic acid suspected of comprising a particular sequence, while a “candidate oligonucleotide” refers to an oligonucleotide suspected of having a property such as comprising a particular sequence, or having the capability to hybridize to a target nucleic acid or to perform in a detection assay. A “candidate detection assay” refers to a detection assay that is suspected of being a valid detection assay.

As used herein, the term “detection panel” refers to a substrate or device containing at least two unique candidate detection assays configured for target detection.

As used herein, the term “valid detection assay” refers to a detection assay that has been shown to accurately predict an association between the detection of a target and a phenotype (e.g. medical condition). Examples of valid detection assays include, but are not limited to, detection assays that, when a target is detected, accurately predict the phenotype medical 95%, 96%, 97%, 98%, 99%, 99.5%, 99.8%, or 99.9% of the time. Other examples of valid detection assays include, but are not limited to, detection assays that quality as and/or are marketed as Analyte-Specific Reagents (i.e. as defined by FDA regulations) or In-Vitro Diagnostics (i.e. approved by the FDA).

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to a delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, allele frequencies, RNA expression levels, protein expression, phenotypes correlating to genotypes, etc. “Allele frequency information” refers to facts or data pertaining allele frequencies, including, but not limited to, allele identities, statistical correlations between the presence of an allele and a characteristic of a subject (e.g., a human subject), the presence or absence of an allele in a individual or population, the percentage likelihood of an allele being present in an individual having one or more particular characteristics, etc.

As used herein, the term “assay validation information” refers to genomic information and/or allele frequency information resulting from processing of test result data (e.g. processing with the aid of a computer). Assay validation information may be used, for example, to identify a particular candidate detection assay as a valid detection assay.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the phrase “non-amplified oligonucleotide detection assay” refers to a detection assay configured to detect the presence or absence of a particular polymorphism (e.g., SNP, repeat sequence, etc.) in a target sequence (e.g. genomic DNA) that has not been amplified (e.g. by PCR), without creating copies of the target sequence. A “non-amplified oligonucloetide detection assay” may, for example, amplify a signal used to indicate the presence or absence of a particular polymorphism in a target sequence, so long as the target sequence is not copied.

### DETAILED DESCRIPTION OF THE INVENTION

The following discussion provides a description of certain preferred illustrative embodiments of the present invention and is not intended to limit the scope of the present invention. For convenience, the discussion focuses on the application of the present invention to the detection of DNA targets, but it should be understood that the methods and systems are intended for use in the development of tools for the analysis of any nucleic acid analyte, e.g., DNA or RNA. Also, for the sake of illustration, the discussion often focuses on the characterization of SNPs using INVADER assay technology. It should be understood that the methods and systems of the present invention are intended for use in detecting other biologically relevant factors using a wide variety of detection assay technologies.

Detection Assay Design

There are a wide variety of detection technologies available for determining the sequence of a target nucleic acid at one or more locations. For example, there are numerous technologies available for detecting the presence or absence of SNPs. Many of these techniques require the use of an oligonucleotide to hybridize to the target. Depending on the assay used, the oligonucleotide is then cleaved, elongated, ligated, disassociated, or otherwise altered, wherein its behavior in the assay is monitored as a means for characterizing the sequence of the target nucleic acid. A number of these technologies are described in detail below.

The present invention provides systems and methods for the design of oligonucleotides for use in detection assays. In particular, the present invention provides systems and methods for the design of oligonucleotides that successfully hybridize to appropriate regions of target nucleic acids (e.g., regions of target nucleic acids that do not contain secondary structure) under the desired reaction conditions (e.g., temperature, buffer conditions, etc.) for the detection assay. The systems and methods also allow for the design of multiple different oligonucleotides (e.g., oligonucleotides that hybridize to different portions of a target nucleic acid or that hybridize to two or more different target nucleic acids) that all function in the detection assay under the same or substantially the same reaction conditions. These systems and methods may also be used to design control samples that work under the experimental reaction conditions. The present invention also provides methods for designing sequences for amplifying the target sequence to be detected (e.g. designing PCR primers for multiplex PCR).

While the systems and methods of the present invention are not limited to any particular detection assay, the following description illustrates the invention when used in conjunction with the INVADER assay (Third Wave Technologies, Madison Wis.; See e.g. U.S. Pat. Nos. 5,846,717; 6,090,543; 6,001,567; 5,985,557; 5,994,069, 6,214,545, 6,210,880, and 6,194,880; Lyamichev et al., Nat. Biotech., 17:292 (1999), Hall et al., PNAS, USA, 97:8272 (2000), Agarwal et al., Diagn. Mol. Pathol. 9:158 [2000], Cooksey et al., Antimicrob. Agents Chemother. 44:1296 [2000], Griffin and Smith, Trends Biotechnol., 18:77 [2000], Griffin and Smith, Analytical Chemistry 72:3298 [2000], Hessner et al., Clin. Chem. 46:1051 [2000], Ledford et al., J. Molec. Diagnostics 2:97 [2000], Lyamichev et al., Biochemistry 39:9523 [2000], Mein et al., Genome Res., 10:330 [2000], Neri et al., Advances in Nucleic Acid and Protein Analysis 3826:117 [2000], Fors et al., Pharmacogenomics 1:219 [2000], Griffin et al., Proc. Natl. Acad. Sci. USA 96:6301 [1999], Kwiatkowski et al., Mol. Diagn. 4:353 [1999], and Ryan et al., Mol. Diagn. 4:135 [1999], Ma et al., J. Biol. Chem., 275:24693 [2000], Reynaldo et al., J. Mol. Biol., 297:511 [2000], and Kaiser et al., J. Biol. Chem., 274:21387 [1999]; and PCT publications WO97/27214, WO98/42873, and WO98/50403, each of which is herein incorporated by reference in their entirety for all purposes) to illustrate preferred features of the present invention) to detect a SNP or other sequence of interest. The INVADER assay provides ease-of-use and sensitivity levels that, when used in conjunction with the systems and methods of the present invention, find use in detection panels, ASRs, and clinical diagnostics. One skilled in the art will appreciate that specific and general features of this illustrative example are generally applicable to other detection assays.

A. INVADER Assay

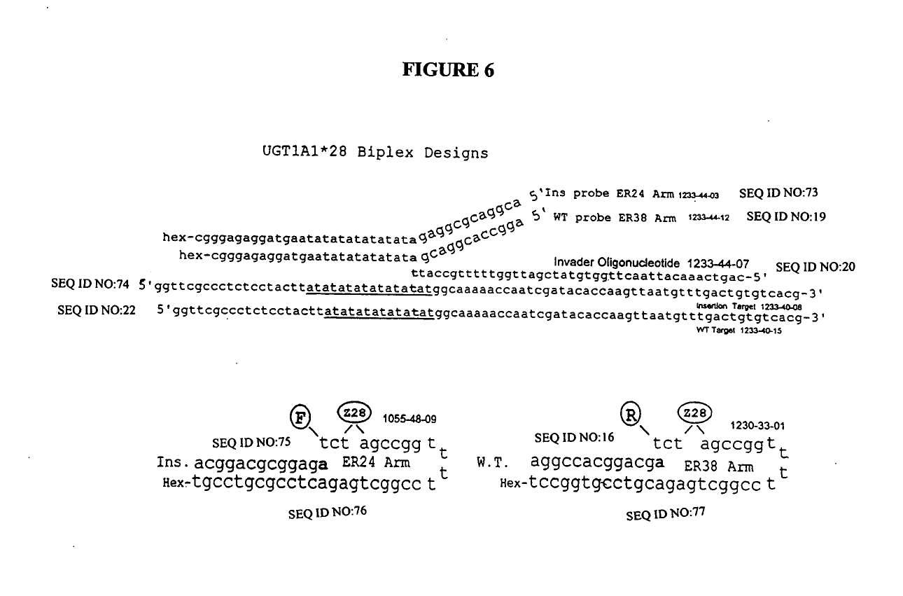

The INVADER assay provides means for forming a nucleic acid cleavage structure that is dependent upon the presence of a target nucleic acid and cleaving the nucleic acid cleavage structure so as to release distinctive cleavage products (See, FIG. 6). 5′ nuclease activity, for example, is used to cleave the target-dependent cleavage structure and the resulting cleavage products are indicative of the presence of specific target nucleic acid sequences in the sample. When two strands of nucleic acid, or oligonucleotides, both hybridize to a target nucleic acid strand such that they form an overlapping invasive cleavage structure, as described below, invasive cleavage can occur. Through the interaction of a cleavage agent (e.g., a 5′ nuclease) and the upstream oligonucleotide, the cleavage agent can be made to cleave the downstream oligonucleotide at an internal site in such a way that a distinctive fragment is produced.

The INVADER assay provides detections assays in which the target nucleic acid is reused or recycled during multiple rounds of hybridization with oligonucleotide probes and cleavage of the probes without the need to use temperature cycling (i.e., for periodic denaturation of target nucleic acid strands) or nucleic acid synthesis (i.e., for the polymerization-based displacement of target or probe nucleic acid strands). When a cleavage reaction is run under conditions in which the probes are continuously replaced on the target strand (e.g. through probe-probe displacement or through an equilibrium between probe/target association and disassociation, or through a combination comprising these mechanisms, (Reynaldo, et al., J. Mol. Biol. 97: 511-520 [2000]), multiple probes can hybridize to the same target, allowing multiple cleavages, and the generation of multiple cleavage products.

The INVADER assay, as well as other assays, may also employ degenerate oligonucleotides (e.g. degenerate INVADER and probe oligonucleotides). For example, standard INVADER oligonucleotides and probes may be randomly changed at one more positions such that a set of degenerate INVADER and/or probe oligonucleotides are produced. Degenerate sets of INVADER and probe oligonucleotides are particularly useful for use in conjunction with target sequences that tend to be heavily mutated (e.g. HIV-1 pol gene). Using such degenerate sets of INVADER and probe oligonucleotides allows the presence of target sequences at a particular location to be detected even if the surrounding sequence no longer represent the wild type or expected sequence.

The INVADER assay technology may be used to quantitate mRNA (e.g. without target amplification). Low variability (3-10% coefficient of variation) provides accurate quantitation of less than two-fold changes in mRNA levels. A biplex FRET-based detection format enables simultaneous quantitation of expression from two genes within the same sample. One of these genes can be an invariant housekeeping gene that is used as the internal standard. Normalizing the signals from the gene of interest with the internal standard provides accurate results and obviates the need for replicate samples. A simple and rapid cell lysate sample preparation method can be used with the mRNA INVADER Assay. The combined features of biplex detection and easy sample preparation make this assay readily adaptable for use in high-throughput applications.

In certain embodiments, the INVADER assay (and other detection assays such as TAQMAN) employ an E-TAG label from Aclara Corporation (e.g. as part of the INVADER oligonucleotide, probe oligonucleotide, or the FRET oligonucleotide). E-TAG labeling is particularly useful in muliplex analysis. E-TAG labeling does not require surface immobilization of affinity agents. E-TAG type labeling is described in U.S. Pat. Nos. 5,858,188; 5,883,211; 5,935,401; 6,007,690; 6,043,036; 6,054,034; 6,056,860; 6,074,827; 6,093,296; 6,103,199; 6,103,537; 6,176,962; and 6,284,113, all of which are herein incorporated by reference. In particularly preferred embodiments, the detection assays of the present invention employ labels described in U.S. Pat. No. 6,001,567, herein incorporated by reference (e.g. fluorescent molecule and linker at the 5′ end of an oligonucleotide).

B. RNA INVADER Assay Design.

For each design method, typically three different INVADER oligonucleotide sets would be designed and screened and the best performing set would be selected as the product assay. If sufficient detection was not achieved with the initial 3-site screen, a redesign method could include moving the cleavage site/accessible site 1 or more nucleotides in either direction and/or lower scoring designs not ordered in the initial process could be ordered and tested.

Integration of the various design methods could involve querying the user or having the user select one or more design methods based on the following examples:
Does the mRNA sequence have significant homology to other genes or gene family members? If yes, should the target sequence be detected exclusively or inclusively?
Is the mRNA sequence one of 2 or more alternatively spliced variants? If yes, should the target sequence be detected exclusively or inclusively?
If closely related sequences or alternatively spliced variants are not identified in the sequence analysis (e.g., via the bioinformatics module), should the candidate assays be designed via the splice site or accessible site method?

Alternatively, as described above, these types of questions can be encoded in an algorithm that would automatically determine the best design strategy based on the automated sequence analysis in the bioinformatics module.

Splice site design. If assay specificity and/or performance requirements do not dictate otherwise, assays can be designed at or near splice junctions to completely preclude the possibility of detecting genomic DNA in a sample. Splice site design involves determining the splice junctions within the mRNA, usually via pairwise alignment of the mRNA sequence with the genomic DNA sequence for that gene, and then locating INVADER assay cleavage sites at or near the splice site. Typically, the INVADER oligonucleotide is positioned on one side of the splice junction and the probe and stacking oligonucleotide (if used) are positioned on the other side. Thus, if the oligonucleotides were bound to genomic DNA, the probe and INVADER oligonucleotides would be separated by the intervening intronic sequences, which would preclude formation of the required overlap substrate for the CLEAVASE enzyme.

Accessible site design. Again, if assay specificity and/or performance requirements do not dictate otherwise, assays can also be designed to accessible sites within the mRNA. Accessible sites are unstructured regions of the RNA and those determined experimentally, for example, using RT-ROL (Allawi et al. RNA 7:314 [2001]), usually correlate well with enhanced INVADER RNA assay performance. Accessible sites can also be determined via in silico analysis. For example, the RNA sequence could be folded in m-Fold software and then analyzed in Oligowalk to determine accessible sites in the RNA. A program could be written to automatically output the accessible sites (defined as a region with negative Overall □G values for an oligonucleotide binding to that region) for the folded RNA. For example, the program could determine when there were 5 or more consecutive nucleotides with Overall □G values of—5 or less, then determine the midpoint of this region, and then output those sites into a file. For example, a 10-base negative □G region encompassing target sequence nucleotides 200-210 would correspond to an accessible site at 205.

In either case, accessible site design could be encoded into the INVADERCREATOR module by method A or B.

Method A

Assays could be designed in reverse of the cleavage site design process. The user would specify the precise position of the 3′ end of the probe within an accessible site and the probe would be built out toward the 5′ end to satisfy the preset Tm requirement. Stacking oligonucleotide (if designing in a stacker format) contributions to the probe's Tm would be determined as the probe was being built and the Invader oligonucleotide would be designed after the program finished the probe or probe/stacker design.

Method B

Another method for accessible site design, using the same probe-building algorithm that is used for cleavage site design methods, is as follows. The user could enter the accessible site and the INVADERCREATOR module could shift a defined number of bases (a default shift could be determined) downstream. For example, 200 could be entered as an accessible site, and INVADERCREATOR module would build a design using the existing algorithm for cleavage site 210 if the shift value was 10. Next to the check box for “Stacker Design” could be a check box for “Accessible Site Design”. Next to this check box could be a field in which the user would designate the number of bases to shift. The current “Cleavage Sites” field could say “Design Sites” to generically encompass either design mode (cleavage sites or accessible sites). Users could have the capability to check one or both boxes (e.g. stacker design and accessible site design, accessible site design only, etc.).

Splice variant design. Splice variant assays can be designed in a variety of ways. An inclusive detection assay could be designed to detect a region of sequence (e.g. a particular exon) present in all variants. A particular splice variant could be detected by designing the assay to a unique splice site (e.g. if a 5 exon gene yields a splice variant that excludes exon 3, the assay could be designed to detect the exon 2-exon 4 splice junction). Since specificity of the INVADER RNA assay is primarily linked to discrimination at the cleavage site, even very small exonic sequences (e.g. a few nucleotides) could be distinguished. In some cases, it may be useful to detect not any one particular mRNA variant but to individually quantitate exons and/or splice junctions in a pool of mRNA variants. The quantitation pattern from this type of INVADER RNA assay analysis may correlate with particular cellular processes or metabolic states.

Discrimination site design. Closely-related sequences would be aligned to the input target sequence and an automated analysis could be performed to identify all sites that contain, for example, two or more adjacent base differences for any one sequence from all others in the alignment. Another automated analysis algorithm could determine regions of homology of sufficient size to accommodate an INVADER oligonucleotide probe set that would inclusively detect all closely-related mRNAs. An output of the location of such double base discrimination sites or regions of homology could be reviewed by the user before accessing the INVADERCREATOR module or automatically designed via input of a batch file.

The present invention is not limited to the use of the INVADERCREATOR software. Indeed, a variety of software programs are contemplated and are commercially available, including, but not limited to GCG Wisconsin Package (Genetics computer Group, Madison, Wis.) and Vector NTI (Informax, Rockville, Md.).

In some embodiments, the present invention provides design parameters for combining multiple nucleic acid detection technologies. For example, in some embodiments, INVADER assays or other assays are used in conjunction with amplified nucleic acid obtained by using the polymerase chain reaction (PCR). In some preferred embodiments, PCR is run simultaneously with other assays.

C. TAQMAN Probe and Primer Design

A number of different strategies can be used to design TaqMan (5′ Nuclease assay) Probes. The following are example of considerations that may be used when designing TAQMAN probes. One consideration is to design PCR primers such that the amplicon size is between 50-150 base pairs. Another consideration is to design PCR primers that have a Tm of around 60° C., with less than 2° C. difference in Tm between forward and reverse primers. Preferred primers have GC % around 40-60% and have three or less consecutive runs of any nucleotide. Preferably, the primers have total lengths of between 18-25 nucleotides in length. PCR Primers are designed to have minimal haripin and minimal dimer formation tendencies (See below). Following selection of the PCR primers, the TAQMAN probe is then chosen from within the amplicon region, and has a Tm of about 10° C. higher than the Tm of the PCR primers (typically, 70° C.). TAQMAN probes should have a 5′ FAM and a 3′ TAMRA (or other labels), and not begin with G. TAQMAN probes may be chosen, for example, by using programs such as Oligo Walk to scan through the amplicon sequence and a probe chosen based upon predicted most stable thermodynamic parameters. Moreover, candidate TAQMAN probes can be eliminated which forms more than three consecutive basepairs with the PCR primers.

D. Sample Preparation Component Design In some embodiments, genomic DNA that contains a target sequence to be analyzed by the detection assay is used as a starting material for the detection assay. In some such embodiments, it may be desirable to amplify the one or more regions of the genomic DNA (e.g., to generate a plurality of target sequences to be detected). The present invention is not limited by the nature of the amplification technology employed. Amplification techniques include, but are not limited to, PCR and the technologies disclosed in U.S. Pat. Nos. 6,345,514 and 6,221,635, as well as foreign patents and applications, EP1113082, WO200146463, WO200146462, JP2001149097, JP 2001136954, and JP2001008660, herein incorporated by reference in their entireties. In certain embodiments, Rubicon OmniPlex technology is employed for sample preparation. Rubicon OmniPlex technology (See e.g., U.S. Pat. No. 6,197,557, herein incorporated by reference in its entirety) reformats naturally occurring chromosomes into new molecules called Plexisomes. Plexisomes represent the complete genome as amplifiable DNA units of equal length that function as a molecular relational database from which the genetic information can be more quickly and accurately recovered. Use of the technology avoids PCR amplification for sample preparation and for genotyping and haplotyping for gene discovery, pharmacogenomics, and diagnostics by providing highly multiplexing and sample amplification. In preferred embodiments, all the various components for running any of these sample preparation methods are included in a kit (e.g. with at least a portion of a detection assay).

Adverse Drug Reactions and Genetic Variation

More than 3 billion prescriptions are written each year in the U.S. alone, effectively preventing or treating illness in hundreds of millions of people. But prescription medications also can cause powerful toxic effects in a patient. These effects are called adverse drug reactions (ADR). Adverse drug reactions can cause serious injury and or even death. Differences in the ways in which individuals utilize and eliminate drugs from their bodies are one of the most important causes of ADRs (MedWatch).

More than 106,000 Americans die—three times as many as are killed in automobile accidents—and an additional 2.1 million are seriously injured every year due to adverse drug reactions. ADRs are the fourth leading cause of death for Americans. Only heart disease, cancer and stroke cause more deaths each year. Seven percent of all hospital patients are affected by serious or fatal ADRs. More than two-thirds of all ADRs occur outside hospitals. Adverse drug reactions are a severe, common and growing cause of death, disability and resource consumption in North America and Europe.

ADRs most commonly occur when the body cannot change a drug quickly enough into a form that it can use and then eliminate. A drug compound goes through a series of many changes as it is being processed in the body, some of which actually may make the drug more toxic before it is changed again. If this toxic form of the drug is not changed or eliminated by the body, it can cause illness, permanent liver damage or even death. Proteins called drug-metabolizing enzymes (DMEs) make these changes as the body processes a drug.

All drugs have the potential to cause ADRs. The most common, however, are central nervous system agents (antidepressants, anticonvulsants, eye and ear preparations, internal analgesics and sedatives), anti-infectious drugs (penicillin and the sulfa antibiotics), anti-cancer drugs and cardiovascular drugs. Cardiovascular drugs alone cause 25 percent of all ADRs.

It is estimated that drug-related anomalies account for nearly 10 percent of all hospital admissions. Drug-related morbidity and mortality in the U.S. is estimated to cost from $76.6 to $136 billion annually.

Irinotecan

An important, and currently available antineoplastic treatment, is called Irinotecan. Irinotecan's chemical formula name is (S)-4,11-diethyl-3,4,12,14-tetrahydro-4-hydroxy-3,14-dioxyo-1H-pyranol[3′,4′:6,7]-indolizino[1,2-b]quinolin-9-y[1,4′-bipeperidine]-1′-carboxylate, monohydrochloride, trihydrate. The empirical formula for Irinotecan is C₃₃H₃₈N₄O₆.HCl.3H₂O and has a molecular weight of 677.19. Irinotecan is currently sold under the name CAMPTOSAR by Pharmacia & Upjohn Corporation. Irinotecan is used to treat cancer (e.g., CAMPTOSAR is approved for colorectal cancer un the United States). The mechanism of action of Irinotecan and its active metabolize SN-38 is preventing topoisomerase I from functioning properly.

Irinotecan (also known as CPT-11) is transformed in vivo by carboxylesterases to an active metabolite called SN-38. SN-38 has about 100-1,000 fold higher antitumor activity than Irinotecan. Irinotecan has been shown to be metabolized by hepatic cytochrome P-450 3A enzymes to a compound called APC, which has a 500 fold weaker antitumor activity compared with SN-38. SN-38 is known to undergo significant bilary excretion and enterohepatic circulation. SN-38 is also subjected to glucuronidation by hepatic uridine diphosphate glucuronosyltransferases (UGTs) to form SN-38G. SN-38G is inactive and is excreted into the urine and bile. Failure to convert SN-38 to SN-38G has been suggested as a cause of diarrehea in patients administered Irinotecan due to an accumulation of SN-38 (See, Lyer et al., J. Clin. Invest., 101 (4), February, 1998, 847-854, herein incorporated by reference).

Clinical studies have shown that Irinotecan was able to significantly improve tumor response rates, time to tumor progression and survival. Irinotecan has shown effectiveness when administered with 5-fluorouracil (5-FU) and leucovorin (LV). Irinotecan is generally administered intravenously.

There are many side effects associated with Irinotecan therapy. One side effect is cholinergic symptoms (e.g. early-onset diarrhea, contraction of pupils, lacrimation, flushing, rhinitis, increased salivation, diaphoresis, and abdominal cramping). Administration of atropine is generally recommended to counteract these symptoms. Another known side effect is late-onset diarrhea, which may be treated with loperamide, IV hydration, and oral antibiotics). Another known side effect is nausea and vomiting. Administration of antiemetic agents on the day of Irinotecan treatment may be used to counteract nausea and vomiting. Finally, another Irinotecan side effect is severe myelosuppression, with deaths due to sepsis being reported.

ii. UGTs, Irinotecan, and Nucleic Acid Screening

UGTs are microsomal enzymes catalyzing the glucuronidation of numerous endogenous and exogenous substrates. Glucuronidation increases the polarity of the substrates to allow them to be better eliminated from the body. The human UGTs are classified into UGT1 and UGT2 families. The UGT1 gene consists of at least 13 unique isoforms with variable exon 1 and common exons 2 to 5. Each of the exons 1 is preceded by its own promoter and differentially spliced to the common exons to produce a unique mature mRNA. The UGT1 family is further classified into multiple isoforms, i.e., UGT1A1, UGT1A3, UGT1A4, up to UGT1A12. The UGT1A1 isoform is responsible for the glucuronidation of bilirubin. The clinically relevant polymorphisms related to genetic abnormalities in UGT1A1 are those associated with familial hyperbilirubinemic syndromes such as Crigler-Najjar syndromes type I (CN-I) and type II (CN-II), and Gilbert's syndrome. CN-I syndrome is rare and is associated with severe unconjugated hyperbilirubinemia. Patients with CN syndromes have absent (CN-I) or reduced (CN-II) UGT1A1 activity with corresponding unconjugated serum bilirubin levels of 15 to >50 mg/dl and 10 to 20 mg/dl, respectively. Gilbert's syndrome is a mild chronic unconjugated hyperbilirubinemia, with serum bilirubin levels usually <3 mg/dl, although higher, lower, and even normal values are not uncommon. A wide variation in the incidences of Gilbert's syndrome has been reported, ranging from 0.5 to 19% in various groups. Gilbert's syndrome is usually associated with homozygosity for the sequence (TA)₇TAA instead of (TA)₆TAA in the promoter region of the UGT1A1 gene, resulting in reduced UGT1A1 expression levels and activity.

In addition to (TA)₆ and (TA)₇ alleles, two new alleles with five and eight TA repeats, i.e., (TA)₅ and (TA)_{g}, have been found (See, Beutler et al., Proc Natl Acad Sci USA, 95:8170-8174, 1998; and DiRienzo et al., Clin Pharmacol Ther 63: 207, 1998, both of which are herin incorporated by reference). These alleles were present in population samples from African ancestry, where they occur at lower frequencies compared with the alleles (TA)₆ and (TA)₇. However, the first Caucasian subject affected by Gilbert's syndrome found to be heterozygous for the (TA)₈ allele has been recently described (See, Iolascon et al., Haematologica 84: 106-109, 1999, herein incorporated by reference). Four alleles of the UGTIAI promoter have been found in 379 individuals sampled at random from 11 aboriginal and admixed populations from different ethnic backgrounds. Allele frequencies vary considerably across ethnic groups, with Asian and American indian populations showing highest frequencies of allele (TA)₆. The frequency of allele (TA)₇ differs significantly between sub-Saharan Africans and Caucasians (See, Hall et al., Pharmacogenetics 9: 591-599, 1999, herein incorporated by reference).

There have been recent reports of heterozygous and homozygous missense mutations in the coding region of UGT1A1 in certain subjects with Gilbert's syndrome who do not have homozygous mutations at the promoter level. The Gly71Arg mutation in the coding region has been shown to result in a 30% (heterozygotes) and 60% (homozygotes) decrease in bilirubin glucuronidating activity.

UGTlA1 polymorphism plays several roles in the metabolism of irinotecan. The example of irinotecan demonstrates how a polymorphism in an inactivating metabolic pathway may affect the therapeutic outcome in cancer chemotherapy. As described above, Irinotecan (CPT-11; 7-ethyl-10-[4-(1-piperidino)-1-piperidino]carbonyloxycamptothecin) is a camptothecin derivative used in the treatment of metastatic colorectal cancer. Irinotecan is a prodrug, since it needs to be activated by systemic carboxylesterases to SN-38 (7-ethyl-10-hydroxycamptothecin) in order to exert its antitumor activity mediated by the inhibition of topoisomerase I. SN-38 undergoes glucuronide conjugation to form the inactive SN-38 glucuronide (SN-38G; 10-O-glucuronyl-SN-38). In addition, two oxidated metabolites of irinotecan have been identified as APC (7-ethyl-10 [4-N-(5-aminopentanoic acid)-1 piperidino]carbonyloxycamptothecin) and NPC [7-ethyl-10-(4-amino-1-piperidino) carbonyloxycamptothecin] formed by CYP3A4 enzyme. APC and NPC have shown weak antitumor activity in vitro.

SN-38 has been associated with the severe diarrhea episodes occurring after irinotecan therapy as a result of the direct enteric injury caused by SN-38. Because it undergoes significant biliary excretion, SN-38 may potentially continue to remain in the gastrointestinal tract, resulting in prolonged diarrhea. The glucuronidation of SN-38 to the inactive SN-38G may protect against irinotecan-induced intestinal toxicities as a result of renal elimination of the more polar SN-38G.

The assessment of pharmacodynamics of SN-38 glucuronidation showed that, with respect to the total irinotecan available in the circulation, patients with relatively low glucuronidation rates had progressive accumulation of SN-38 leading to toxicity (Gupta et al., Cancer Res 54: 3723-3725, 1994). A genetic predisposition to the metabolism of irinotecan may be critical in patients with reduced UGT1A1 activity (Iyer et al., J Clin Invest 101:847-854, 1998, herein incorporated by reference). As the distinction between mild instances of the syndrome and normal condition is sometimes difficult, Gilbert's syndrome remains often undiagnosed.

Genotyping of UGT1A1 promoter mutations may predict the functional activity of UGT1 A1. A correlation analysis with the corresponding phenotyping results is necessary to demonstrate the validity of the genotyping procedure. Iyer et al., (Clin Pharmacol Ther 65: 576-582, 1999, herein incorporated by reference) recently showed a good concordance between UGT1 A1 promoter genotype and in vitro glucuronidation of SN-38 in human livers of Caucasian origin. SN-38 glucuronidation rates were significantly lower in homozygotes (TA)₇/(TA)₇ and heterozygotes (TA)₆/(TA)₇ when compared with the wild-type genotype (TA)₆/(TA)₆.

A high variability in SN-38 glucuronidation reported in liver samples from populations of African descent (Iyer et al., Clin Pharmacol Ther 65: 197, 1999, herein incorporated by reference) can be explained by the presence of five and eight TA repeats, i.e., (TA)₅ and (TA)₈, in the UGT1A1 promoter (see, Beutler et al., 1998; and DiRienzo et al., 1998). According to this evidence, greater and lesser glucuronidating activity of SN-38 has been found in (TA)₅ and (TA)₈ liver samples, respectively (see, Iyer et al., 1999). UGT1A1 activity is inversely related to the number of TA repeats, since the transcriptional activity of the promoter decreases with the progressive increase in the number of TA repeats (see, Beutler et al., 1998). These new alleles indicate that up to 10 genotypes may exist at the TATAA element, probably resulting in different phenotypes with regard to bilirubin conjugation and irinotecan pharmacokinetics. Based upon in vitro phenotyping of UGT1A1 activity in livers, homozygotes for (TA)₇ and heterozygotes (TA)₆/(TA)₇ might be expected to have at least a 50 and 25% decrease in SN-38 glucuronidating activity, respectively (see, Iyer et al., 1999). A significantly impaired ability to glucuronidate SN-38 has been found in one patient genotyped as (TA)₇ homozygote (Ando et al., Ann Oncol 9: 845-847, 1998, herein incorporated by reference). Consequently, appropriate irinotecan dose reductions may be necessary in homozygotes for (TA)₇ and heterozygotes (TA)₆/(TA)₇.

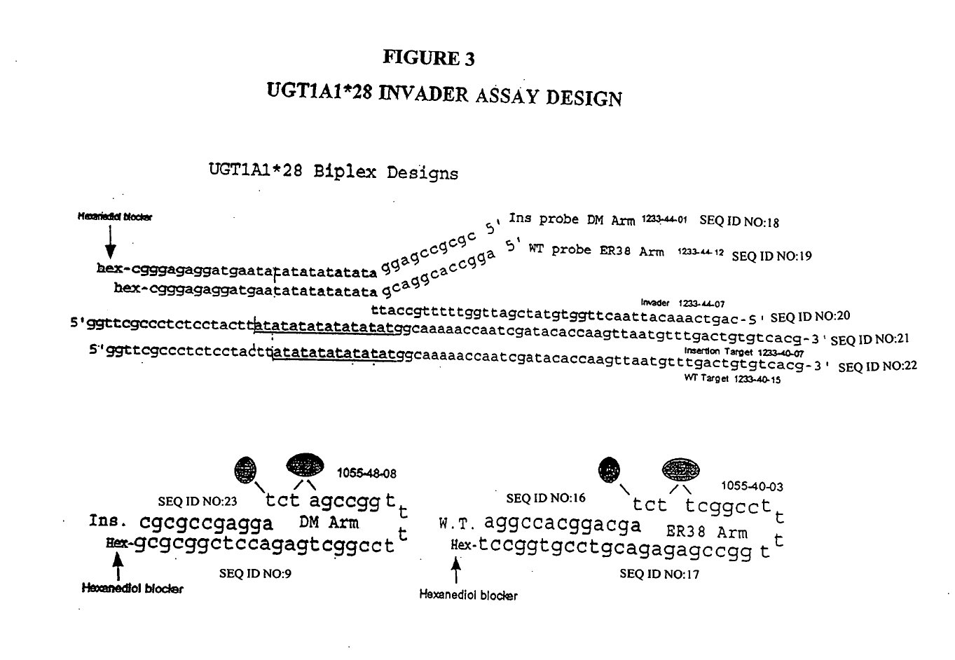

As mentioned above, Irinotecan is known to metabolized by UGT's. As such, the present invention provides systems and methods for screening subjects that are candidates for Irinotecan administration, or patients already taking Irinotecan. Any type of detection assay may be employed including, but not limited to; a hybridization assay, a TAQMAN assay, or an invasive cleavage assay (e.g. INVADER assay), a mass spectroscopy based assay, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. The detection assay may be configured to detect various polymorphism of UGT1 A1, and/or the wild type allele, since wild type UGT1A1 is known to properly metabolize SN-38 to SN-38G. The detection assay may be configured to detectin TA repeats in the UGT1A1 promoter region (See, e.g., Invader assays in FIGS. 3, 6 and 7). The detection assay may also be configured to detect cytochrome P-450 3A enzyme polymorphims.

The human wild type UGT1A1 sequence is under accession number NM_{—}000463. There are many polymorphisms in UGT1A1. Below, in Table 1, is a list of fifteen polymorphisms in UGT1A1, along with a reference describing these polymorphism.

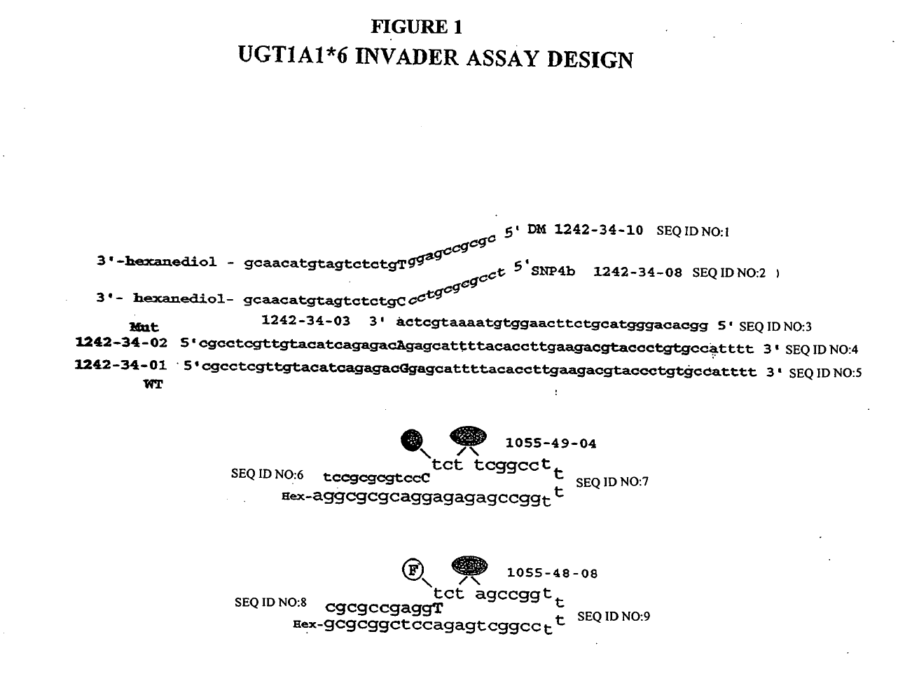

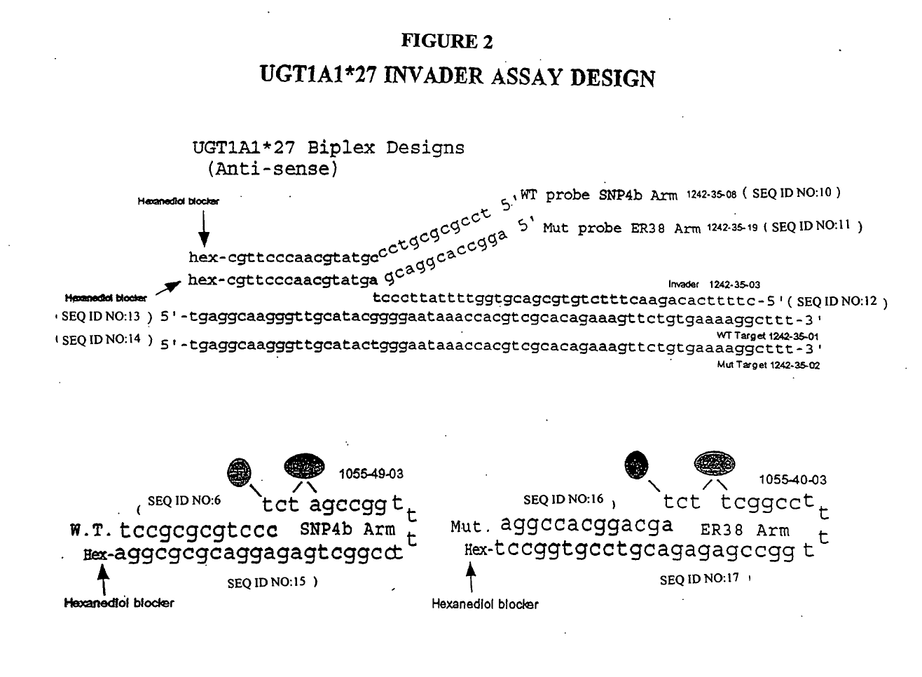

TABLE 1

| | |
|---|---|
| 1. | UGT1A1, 13-BP DEL, EX2, see, Ritter et al., J. Clin. Invest. 90: 150-155, 1992, |
| | hereby incorporated by reference. This variant has been designated UGT1A1*2. |
| 2. | UGT1A1, Ser376Phe (C to T transition in Exon 4, see, Bosma, et al., FASEB J. 6: |
| | 2859-2863, 1992, hereby incorporated by reference). This variant has been designated |
| | UGT1A1*3. |
| 3. | UGT1A1, Gln 331Ter (C to T transition, see, Bosma, et al., FASEB J. 6: 2859-2863, |
| | 1992, hereby incorporated by reference). This variant has been designated UGT1A1*5. |
| 4. | UGT1A1, Arg 341Ter (nonsense CGA to TGA mutation, see, Moghrabi et al., |
| | Genomics 18: 171-173, 1993, hereby incorporated by reference). This variant has been |
| | designated UGT1A1*10. |
| 5. | UGT1A1, Gln331Arg (A to G transition, see Moghrabi et al., Genomics 18: 171-173, |
| | 1993, hereby incorporated by reference). This variant has been designated UGT1A1*9. |
| 6. | UGT1A1, Phe170Del (See, Ritter et al., J. Biol. Chem. 268: 23573-23579, 1993, |
| | hereby incorporated by reference). This variant has been designated UGT1A1*13. |
| 7. | UGT1A1, Gly309Glu (G to Transition in codon 309, see, Erps et al., J. Clin. |
| | Invest. 93: 564-570, 1994, hereby incorporated by reference). This variant has been designated |
| | UGT1A1*11. |
| 8. | UGT1A1, 840C to A, Cys-Ter (See, Aono et al., Pediat. Res. 35: 629-632, 1994, |
| | hereby incorporated by reference). This variant has been designated UGT1A1*25. |
| 9. | UGT1A1, Pro229Gln (C to A transition at nucleotide 686, See, Koiwai et al., |
| | Hum. Molec. Genet. 4: 1183-1186, 1995, hereby incorporated by reference. This variant has |
| | been designated UGT1A1*27. Also, see FIG. 2 providing an exemplary INVADER detection |
| | assay design to detect this polymorphism. |
| 10. | UGT1A1, 2-BP insertion “TA” in TATA promoter region (See, Bosma et al., |
| | New Eng. J. Med. 333: 1171-1175, 1995, hereby incorporated by reference. This variant has |
| | been designated UGT1A1*28. Also, see FIG. 3, providing an exemplary INVADER detection |
| | assay design to detect this polymorphism. |
| 11. | UGT1A1, 1-BP insertion, 470T (See, Rosatelli et al., J. Med. Genet. 34: |
| | 122-125, 1997, hereby incorporated by reference). |
| 12. | UGT1A1, IVS1, G-C +1 (G to C mutation at the splice donor site in intron |
| | between exon 1 and exon 2, see, Gantla et al., Am. J. Hum. Genet. 62: |
| | 585-592, 1998, hereby incorporated by reference). |
| 13. | UGT1A1, 145C-T (See, Gantla et al., Am. J. Hum. Genet. 62: |
| | 585-592, 1998, hereby incorporated by reference). |
| 14. | UGT1A1, IVS3, A-G, −2 (See, Gantla et al., Am. J. Hum. Genet. 62: |
| | 585-592, 1998, hereby incorporated by reference). |
| 15. | UGT1A1, Gly71Arg (A to G change at nucleotide 211 in exon 1, see, Akaba et |
| | al., Biochem. Molec. Biol. Int. 46: 21-26, 1998, hereby incorporated by reference). Also, see |
| | FIG. 1, providing an exemplary INVADER detection assay design to detect this |
| | polymorphism. |

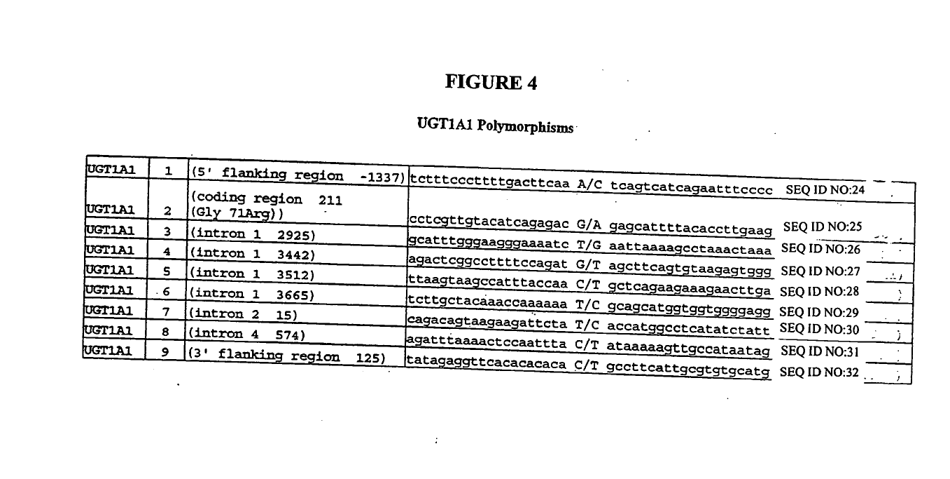

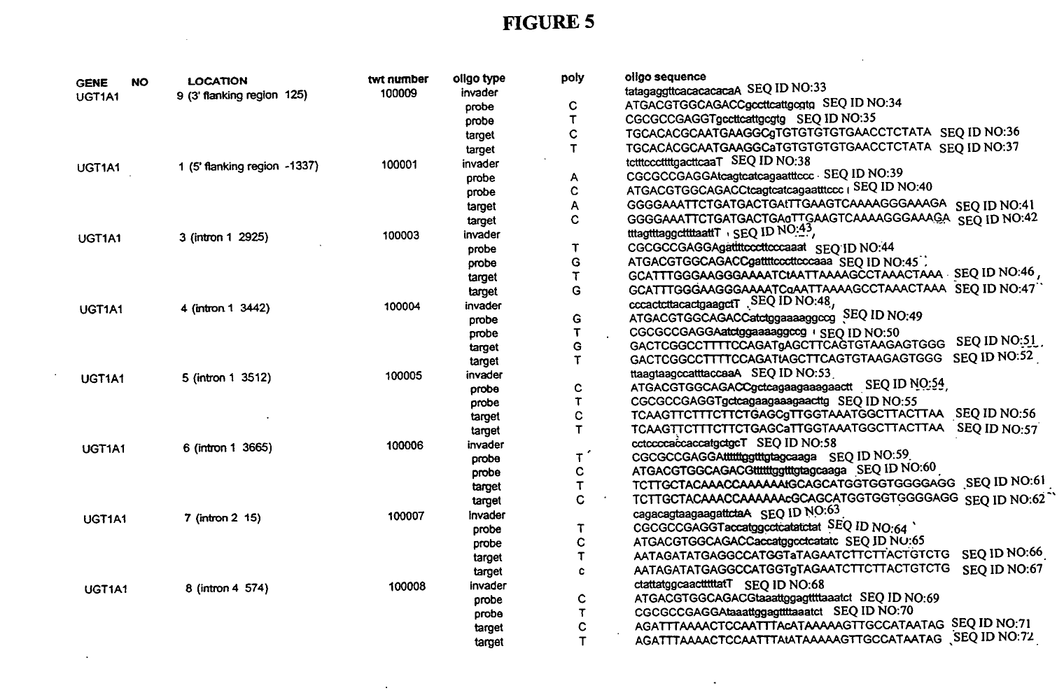

Another set of nine polymorphisms in UGT1A1 is provided in FIG. 4 (see, e.g., U.S. patent application Ser. No. 10/035,833, Table 1, which is incorporated herein by reference). Exemplary detection assays (INVADER assays) for these nine polymorphisms are provided in FIG. 5, although any type of detection assay may be employed to detect these polymorphisms.

In some embodiments, the present invention provides methods for selecting therapy for a subject, comprising; a) providing; i) a sample from the subject, and ii) a detection assay configured to detect a polymorphism in a gene sequence associated with Irinotecan safety or efficacy, b) contacting the sample with the detection assay under conditions such that the presence or absence of the polymorphism in the gene sequence is determined, and c) identifying the subject as suitable for treatment with Irinotecan based on the absence of the polymorphism in the gene sequence; or identifying the subject as not suitable for treatment with Irinotecan based on the presence of the polymorphism in the gene sequence. In other embodiments, the methods further comprise step d) administering Irinotecan to the subject identified as suitable for treatment with Irinotecan. In certain embodiments, the methods further comprise step d) informing the subject that they have been identified as not suitable for treatment with Irinotecan.

In some embodiments, the gene sequence associated with Irintoecan safety or efficacy is UGT1A1 (e.g. human UGT1A1). In other embodiments, the polymorphism in the gene associated with Irinotecan safety or efficacy is selected from a UGT1A1 polymorphism listed in Table 1, or a UGT1A1 polymorphism listed in FIG. 4. In particular embodiments, the gene sequence associated with Irinotecan safety or efficacy is an P-450 3A enzyme. In preferred embodimnts, the polymorphism is a repeat sequence (e.g. TA repeat) in the promoter region of the UGTlA1 gene (e.g. a 5 repeat, 6 repeat, 7 repeat, or 8 repeat). In other preferred embodiments, the repeats are detected with the INVADER assay (See, e.g., FIGS. 3, 6, and 7).

In certain embodiments, the subject has been diagnosed with cancer. In other embodiments, the cancer is colorectal cancer. In additional embodiments, the sample from the subject is a blood sample, urine sample, semen sample, skin sample, or hair sample. In some embodiments, the detection assay is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, the detection assay is an INVADER detection assay. In particularly preferred embodiments, the INVADER detection assay is selected from those shown in FIG. 5.

In certain embodiments, the sample is also screened with a detection assay to determine if the subject will benefit from a second drug that counteract side-effects of Irinotecan administration (exampled of second drugs include, but are not limited to, atropine, loperamide, and antimetics). In other embodiments, the side effects are selected from early-onset diarrhea, contraction of pupils, lacrimation, flushing, rhinitis, increased salivation, diaphoresis, abdominal cramping, late-onset diarrhea, nausea, vomiting, myelosuppression, and sepsis. In certain embodiments, the subject is administered Irinotecan and a second drug to counteract the side effects of the Irinotecan administration.

In some embodiments, the detection assay is located on a panel (e.g. a detection panel configured to detect at least one UGT1A1 polymorphism shown in FIG. 4). In other embodiments, the conditions in the contacting step comprises performing a mutiplexed PCR amplification reaction.

In certain embodiments, the present invention provides methods for selecting therapy for a subject, comprising; a) providing; i) a sample from the subject, and ii) a detection panel comprising at least two unique detection assays, wherein each of the at least two unique detection assays is configured to detect a polymorphism in a gene sequence associated with Irinotecan safety or efficacy, b) contacting the sample with the detection panel under conditions such that each of the at least two unique detection assays reveals the presence or absence of a polymorphism, and c) identifying the subject as suitable for treatment with Irinotecan based on the absence of polymorphisms detected by the at least two detection assays, or identifying the subject as not suitable for treatment with Irinotecan based on the presence of at least one polymorphism detected by the at least two detection assays. In some embodiments, the methods further comprise step d) administering Irinotecan to the subject identified as suitable for treatment with Irenotecan. In other embodiments, the methods further comprise step d) informing the subject that they have been identified as not suitable for treatment with Irenotecan.

In particular embodiments, each of the at least two unique detection assays is configured to detect a polymorphism in the UGT1A1 gene. In preferred embodiments, each of the at least two unique detection assays is configured to detect a polymorphism selected from a UGT1A1 polymorphism listed in Table 1, or a UGTlA1 polymorphism listed in FIG. 4. In particularly preferred embodiments, at least one of the detection assays is selected from a UGT1A1 polymorphism listed in FIG. 4. In other embodiments, at least one of the detection assay is configured to detect a polymorphism is an P-450 3A enzyme.

In certain embodiments, the subject has been diagnosed with cancer. In other embodiments, the cancer is colorectal cancer. In some embodiments, the sample from the subject is a blood sample, urine sample, semen sample, skin sample, or hair sample. In certain embodiments, at least one of the at least two detection assays is selected from a TAQMAN assay, or an INVADER assay, a polymerase chain reaction assay, a rolling circle extension assay, a sequencing assay, a hybridization assay employing a probe complementary to the polymorphism, a bead array assay, a primer extension assay, an enzyme mismatch cleavage assay, a branched hybridization assay, a NASBA assay, a molecular beacon assay, a cycling probe assay, a ligase chain reaction assay, and a sandwich hybridization assay. In preferred embodiments, at least one of the detection assays is an INVADER detection assay. In particularly preferred embodiments, the INVADER detection assay is selected from those shown in FIG. 5.

In certain embodiments, the sample is also screened with a detection assay to determine if the subject will benefit from a second drug that counteracts side effects of Irinotecan administration. Examples of second drugs include, but are not limited to, atropine, loperamide, and antimetics. In other embodiments, the side effects are selected from early-onset diarrhea, contraction of pupils, lacrimation, flushing, rhinitis, increased salivation, diaphoresis, abdominal cramping, late-onset diarrhea, nausea, vomiting, myelosuppression, and sepsis.

In particular embodiments, the subject is administered irinotecan and a second drug to counteract the side effects of the Irinotecan administration. In other embodiments, the conditions in the contacting step comprises performing a mutiplexed PCR amplification reaction.

In some embodiments, the present invention provides kits comprising; a) a detection assay configured to detect a polymorphism in a gene sequence associated with Irinotecan safety or efficacy, and b) written component, wherein the written component comprises instructions for identifying if a subject is suitable for treatment with Irinotecan based on the results of employing the detection assay on a sample from the patient. In other embodiments, the present invention provides kits comprising; a) a detection assay configured to detect a polymorphism in a gene sequence associated with Irinotecan safety or efficacy, and b) a composition comprising Irinotecan.

In certain embodiments, the present invention provides methods of marketing, comprising; advertising the sale of Irinotecan and a detection assay configured to detect a polymorphism in a gene sequence associated with Irinotecan safety or efficacy together. In other embodiments, the present invention provides methods comprising; a) designing a detection assay to detect a polymorphism associated with Irinotecan safety or efficacy in a subject, and b) drafting a patent application based on the combination of the detection assay and drug. In other embodiments, the methods further comprise filing the patent application in the United States Patent and Trademark Office. In some embodiments, the present invention provides a patent resulting from the above methods.

The present invention provides methods for detecting polymorphisms in genes affecting the action of therapeutic agents. In some embodiments, detection of a single polymorphism, or any combinations of polymorphisms can be performed on amplification products, including cDNA, run-off transcripts, genomic DNA or RNA. In another embodiment, the method includes detection of polymorphisms known to occur more frequently in a particular ethnic group, gender, or age group, and which are associated with therapeutic dosing decisions for, or adverse reactions to a particular therapeutic agent. In a preferred embodiment, a universal kit would be used to screen for any polymorphism that correlates with an adverse reaction to, a therapeutic agent for, any ethnicity, gender, or age. In another embodiment, the universal test screens for polymorphisms associated with an adverse reaction or therapeutic dosing decision wherein some subset of the universal kit polymorphisms would be used based on phenotypic information such as ethnicity, gender, or age any combination of phenotypic information. In one embodiment, the universal test could be used to screen for all UGT1A1 polymorphisms before drug or therapeutic agent administration. In one preferred embodiment, the universal test could screen for all UGT1A1 polymorphisms before administering irinotecan. In another embodiment, the universal test could screen for all UGT1A1 polymorphisms and additional polymorphisms in other genes that correlated with the benefit or outcome for irinotecan therapy.

Another embodiment of the present invention includes entering phenotypic information such as ethnicity, gender, age, height, weight, etc. into a therapy analysis algorithm, wherein said algorithm may utilize any one or combination of these phenotypic parameters along with any one or combination of genotypic results included in the universal test to determine the patient's therapy. It is envisioned that one or more polymorphisms may provide a means for sub-selection of data from another set of polymorphisms that could be utilized for therapeutic decisions such as whether to use a particular therapeutic agent and at what dosing level or if another therapeutic agent is recommended. In another embodiment, one or more phenotypic parameters may provide a means for sub-selection of data from a set of polymorphisms that could be utilized for therapeutic decisions.

In some embodiments, the present invention provides systems for manufacturing and/or selling pharmacogenetic detection assays, comprising: a) a pharmacogenetic detection assay production component for creating a UGT polymorphism detecting pharmacogenetic oligonucleotide detection assay; b) a pharmacogenetic detection assay quality control component; and c) a label generator, wherein the label generator comprises a device for providing indicia on a package or package insert related to the UGT polymorphism detecting pharmacogenetic detection assay, wherein the indicia is selected from the group consisting of intended use indicia, patient population indicia, proprietary name indicia, established name indicia, quantity indicia, concentration indicia, source indicia, measure of activity indicia, warning indicia, precaution indicia, storage instruction indicia, reconsitution indicia, expiration date indicia, observable indication of alteration indicia, net quantity of contents indicia, number of tests indicia, manufacturer indicia, packer indicia, distributor indicia, lot number indicia, control number indicia, chemical principle indicia, physiological principle indicia, biological principle indicia, mixing instruction indicia, sample preparation indicia, use of instrumentation indicia, calibration indicia, specimen collection indicia, known interfering substances indicia, step by step outline of recommended procedures from reception of specimen to result indicia, indicia indicative for improving performance, indicia indicative for improving accuracy, list of materials indicia, amount indicia, time indicia used to assure accurate results, positive control indicia, negative control indicia, indicia explaining the calculation of an unknown, formula indicia, limitation of procedure indicia, additional testing indicia, range of expected value indicia, specificity indicia, sensitivity indicia, pertinent reference indicia, batch indicia, and date of issuance of last revision of label indicia.

In some embodiments, the storage instruction indicia is selected from the group consisting of temperature indicia, and humidity indicia. In other embodiments, the system further comprises a device for providing multiple container packaging for the pharmacogenetic oligonucleotide detection assay. In other embodiments, the quality control component comprises an electronic document control component. In particular embodiments, the quality control component further comprises a purchasing control component.

In some embodiments, the quality control component further comprises a vendor ranking component. In other embodiments, the vendor ranking component comprises a vendor quality ranking component. In certain embodiments, the system further comprises a database of acceptable supplier, contractors, and consultants. In particular embodiments, the quality control component comprises a database comprising electronic purchasing documents. In other embodiments, the system further comprises a product identifier component. In some embodiments, the product identifier component comprises a system for identifying a pharmacogenetic olionucleotide detection assay or components thereof through a stage, the stage selected from the group consisting of a receipt stage, production stage, distribution stage, and installation stage. In certain embodiments, the product identifier component comprises a fail safe anti-mix up module. In some embodiments, the quality control component further comprises a contamination control component.

In particular embodiments, the quality control component comprises validated computer software. In other embodiments, the quality control component further comprises electronic calibration records for one or more components of the system. In some embodiments, the quality control component further comprises a non-conforming pharmacogenetic oligonucleotide detection assay rejection component. In other embodiments, the non-conforming product rejection component further comprises a system for evaluation, segregation and disposition of non-conforming pharmacogenetic oligonucleotide detection assay rejection component. In some embodiments, the production component communicates with the quality control component. In further embodiments, the communication comprises a non-conformance notifier.

In other embodiments, the quality control component further comprises statistical routines to detect a quality problem with the pharmacogenetic oligonucleotide detection assay. In certain embodiments, the system further comprises a pharmacogenetic oligonucleotide detection assay device master recorder. In some embodiments, the system further comprises a pharmacogenetic oligonucleotide detection assay device history recorder. In additional embodiments, the device history recorder comprises data of a detection assay or batch manufacture date, quantity data, quality data, acceptance record data, primary identification label data, and control number data. In other embodiments, the system further comprises a quality system recorder. In yet other embodiments, the system further comprises a complaint file recorder.

In particular embodiments, the system further comprises a pharmacogenetic oligonucleotide detection assay tracker. In other embodiments, the pharmacogenetic detection assay is a detection assay capable of detecting one or more TA repeats in a promoter of the gene. In some embodiments, the pharmacogenetic detection assay is a detection assay capable of detecting five or more TA repeats in a promoter of the gene. In other embodiments, the pharmacogenetic detection assay is a detection assay capable of detecting eight or more TA repeats in a promoter of the gene.

In some embodiments, the pharmacogenetic detection assay comprises a plurality of detection assays capable of detecting gene expression or more than one polymorphisms across different ethnic groups. In additional embodiments, the different ethnic groups are selected from the group consisting of an African American ethnic group, an asian ethnic group, a hispanic ethnic group, and a Caucasian ethnic group.

In other embodiments, the more than one polymorphisms in UGT1A1 are selected from the group of one or more TA repeats in a promoter region of the gene. In some embodiments, the production component is configured to produce or inventory substantially similar batch quantities of two or more detection assays or detection assay components. In particular embodiments, the detection assays are selected from the group consisting of detection assays configured to detect TA repeats in a promoter region of the gene, in combination with one or more exonic polymorphisms. In other embodiments, the pharmacogenetic detection assay is a detection assay capable of detecting gene expression of a UGT1A1 gene, and one or more TA repeats in a promoter of the gene, and one or more exonic polymorphisms in the gene. In further embodiments, the pharmacogenetic detection assay is a detection assay capable of detecting gene expression of a UGT1A1 gene comprising one or more polymorphisms, the polymorphisms selected from the group consisting of promoter region polymorphisms and exonic polymorphisms. In some embodiments, the pharmacogenetic detection assay is a detection assay capable of detecting gene expression of a gene. In other embodiments, the pharmacogenetic detection assay is a detection assay capable of detecting gene expression of a gene across more than one ethnic group. In particular embodiments, the pharmacogenetic detection assay is a detection assay capable of detecting gene expression of a gene across all ethnic groups.

In some embodiments, the detection assay comprises a hybridization assay. In other embodiments, the detection assay comprises a TAQMAN assay. In other embodiments, the detection assay comprises an invasive cleavage assay. In further embodiments, the detection assay comprises mass spectroscopy. In other embodiments, the detection assay comprises microarray. In other embodiments, the detection assay comprises a polymerase chain reaction. In further embodiments, the detection assay comprises a rolling circle extension assay. In some embodiments, the detection assay comprises a sequencing assay.

In particular embodiments, the detection assay comprises a hybridization assay employing a probe complementary to a polymorphism. In other embodiments, the detection assay comprises a bead array assay. In some embodiments, the detection assay comprises a primer extension assay. In additional embodiments, the detection assay comprises an enzyme mismatch cleavage assay. In particular embodiments, the detection assay comprises a branched hybridization assay. In other embodiments, the detection assay comprises a NASBA assay. In further embodiments, the detection assay comprises a molecular beacon assay. In still other embodiments, the detection assay comprises a cycling probe assay. In some embodiments, the detection assay comprises a ligase chain reaction assay. In other embodiments, the detection step comprises a sandwich hybridization assay.

In some embodiments, the system further comprises a drug production or drug inventory level monitoring device for a drug, whereby production or inventory of the pharmacogenetic detection assay is adjusted upward or downward based upon data transmitted from the monitoring device. In further embodiments, the drug is irinotecan or a derivative thereof, and in which the pharmacogenetic detection assay is capable of determining the presence or absense of one or more drug metabolism markers, the markers selected from the group consisting of UGT 1A1 promoter region polymorphisms, and UGT 1A1 exonic polymorphisms.

In some embodiments, the present invention provides pharmacogenetic detection assay kits (e.g. created via the systems above). In further embodiments, the detection assay comprises a hybridization assay. In other embodiments, the detection assay comprises a TAQMAN assay. In some embodiments, the detection assay comprises an invasive cleavage assay. In other embodiments, the detection assay comprises mass spectroscopy, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, or a sandwich hybridization assay.

The present invention also provides pharmacogenetic detection assay kits created by the system described above in which the pharmacogenetic detection assay is capable of detecting one or more polymorphisms in UGT1A1. In some embodiments, the polymorphisms are associated with metabolism of camptothecin, or a derivative thereof. In other embodiments, the camptothecin derivative is Topotecan or Irinotecan. In some embodiments, the camptothecin derivative is Irinotecan. In additional embodiments, the detection assay comprises a hybridization assay. In other embodiments, the detection assay comprises a TAQMAN assay, an invasive cleavage assay, mass spectroscopy, a microarray, a polymerase chain reaction, a rolling circle extension assay, a sequencing assay, a hybridization assay employing a probe complementary to a polymorphism, a bead array assay, a primer extension assay, an enzyme mismatch cleavage assay, a branched hybridization assay, a NASBA assay, a molecular beacon assay, a cycling probe assay, a ligase chain reaction assay, or a sandwich hybridization assay.

In other embodiments, the kit further comprises a drug. In some embodiments, the drug comprises camptothecin, or a camptothecin derivative. In some embodiments, the camptothecin derivative is Topotecan or Irinotecan. In certain embodiments, the camptothecin derivative is Irinotecan. In some embodiments, the drug is irinotecan or a derivative thereof, and in which the pharmacogenetic detection assay is capable of determining the presence or absence of one or more drug metabolism markers, the markers selected from the group consisting of UGT 1A1 *6, *7, *27, *28, and *29.

In other embodiments, the present invention provides universal pharmacogenetic detection assay kits, in which the pharmacogenetic detection kit members are capable of detecting two or more polymorphisms prevalent in three or more ethnic groups. In some embodiments, the ethnic groups are selected from the group consisting of African Americans, Caucasians, Asians, Europeans, and Indian Americans.

In some embodiments, the present invention provides pharmacogenetic detection assay kits, in which the pharmacogenetic detection kit members are capable of detecting polymorphisms in a UGT gene, the kit capable of detecting polymorphisms common in more than one ethnic group and, in which the polymorphisms have a threshold allelle frequency. In certain embodiments, the threshold allelle frequency is greater than about one percent, and in which the kit is capable or detecting two or more polymorphisms common in a first ethnic group, and two or more polymorphisms common in a second ethnic group. In other embodiments, the threshold allelle frequency is greater than about three percent, and in which the kit is capable of detecting X number of polymorphisms common in a first ethnic group, where X is an interger greater than or equal to two, in which the kit is capable or detecting Y number of polymorphisms common in a second ethnic group, where Y is an interger greater than or equal to two, and in which the kit is capable or detecting Z number of polymorphisms common in a third or more ethnic groups, where Z is an interger greater than or equal to two.

In certain embodiments, the threshold allelle frequency is greater than about five percent. In other embodiments, the threshold allelle frequency is greater than about ten percent. In still other embodiments, the threshold allelle frequency is in the range of about 1 percent to about 95 percent. In some embodiments, the polymorphisms are associated with metabolism of camptothecin, or a derivative thereof. In further embodiments, the camptothecin derivative is Topotecan or Irinotecan. In other embodiments, the camptothecin derivative is Irinotecan.

In some embodiments, the kit further comprises a drug. In other embodiments, the drug comprises camptothecin, or a camptothecin derivative. In certain embodiments, the camptothecin derivative is Topotecan or Irinotecan. In additional embodiments, the camptothecin derivative is Irinotecan.

In some embodiments, the present invention provides pharmacogenetic detection assay kits in which the pharmacogenetic detection kit members are capable of detecting polymorphisms related to more than one ethnic group and, in which the polymorphisms have a threshold allelle frequency.

In some embodiments, the present invention provides methods for detecting polymorphisms in a uridine diphosphate glucuronosyl transferase (UGT) gene promoter comprising determining the presence or absence of at least five or greater (TA) repeats in the promoter with a non-amplified oligonucleotide detection assay. In certain embodiments, the methods comprise the steps of (a) obtaining DNA or RNA from an individual; and, (b) determining the number of TA repeats in the promoter. In particular embodiments, the promoter is the UGT1A1 promoter. In certain embodiments, the method further comprises amplifying DNA other than all or part of the UGT1A1 promoter DNA in a multiplexed amplification step. In particular embodiments, the promoter has a genotype selected from the group consisting of [TA]₅/[TA]₅, [TA]₅/[TA]₆, [TA]₅/[TA]₇, [TA]₅/[TA]₈, [TA]₆/[TA]₈, [TA]₇/[TA]₈ and [TA]₈/[TA]₈.

In certain embodiments, the present invention provides methods for optimizing drug dosages for a patient wherein the drugs are glucuronidated by a uridine diphosphate glucuronosyltransferase (UGT), determining the number of thymidine-adenine (TA) repeats in a promoter of the UGT gene by a non-amplified oligonucleotide detection assay, and up or down dosing the patient based upon the determination. In some embodiments, the non-amplified oligonucleotide detection assay is capable of detecting polymorphisms prevalent above a threshold allele frequency in more than one ethnic group. In additional embodiments, the threshold allelle frequency is greater than about one percent. In other embodiments, the threshold allelle frequency is greater than about three percent. In further embodiments, the threshold allelle frequency is greater than about five percent. In other embodiments, the threshold allelle frequency is greater than about ten percent. In some embodiments, the threshold allelle frequency is in the range of about 1 percent to about 95 percent.

In some embodiments, the non-amplified oligonucleotide detection assay is capable of detecting polymorphisms prevalent in Asians, African Americans, Hispanics and Caucasians. In other embodiments, the promoter has a genotype selected from the group consisting of [TA]₅/[TA]₅, [TA]₅/[TA]₆, [TA]₅/[TA]₇, [TA]₅/[TA]₈, [TA]₆/[TA]₈, [TA]₇/[TA]₈ and [TA]₈/[TA]₈.

In certain embodiments, the present invention provides methods for optimizing drug dosages for a patient wherein the drugs are glucuronidated by a uridine diphosphate glucuronosyltransferase (UGT), determining the number of thymidine-adenine (TA) repeats in a promoter of a UGT gene by a universal oligonucleotide detection assay capable of detecting two or more genetic polymorphisms across two or more ethnic groups. In other embodiments, the method further comprises up or down dosing the patient based upon the determination. In particular embodiments, the universal oligonucleotide detection assay is capable of detecting three or more genetic polymorphisms across three or more ethnic groups. In some embodiments, the method further comprises monitoring gene expression of the UGT gene.

In some embodiments, the present invention provides methods for optimizing drug dosages for a patient wherein the drugs are glucuronidated by a uridine diphosphate glucuronosyltransferase (UGT), determining gene expression of the UGT gene by an oligonucleotide detection assay capable of detecting gene expression of the UGT gene. In other embodiments, the method further comprises up or down dosing the patient based upon the determination. In some embodiments, the oligonucleotide detection assay is capable of detecting gene expression in two or more ethnic groups.

In some embodiments, the present invention provides kits for optimizing drug dosages for a patient wherein the drugs are glucuronidated by a uridine diphosphate glucuronosyltransferase (UGT), comprising oligonucleotide detection assay components capable of detecting gene expression of the UGT gene. In other embodiments, the oligonucleotide detection assay components are capable of detecting gene expression in two or more ethnic groups. In some embodiments, the kit further comprises a drug. In particular embodiments, the drug comprises camptothecin, or a camptothecin derivative. In some embodiments, the camptothecin derivative is Topotecan or Irinotecan. In certain embodiments, the gene further comprises a gene promoter, the promoter of the gene having a genotype selected from the group consisting of [TA]5/[TA]5, [TA]5/[TA]6, [TA]5/[TA]7, [TA]5/[TA]8, [TA]6/[TA]8, [TA]7/[TA]8 and [TA]8/[TA]8.

Detection of UGT1A1 Dinucleotide Repeat Polymorphism *28

The hepatic uridine diphosphate glucuronosyltransferase (UGT) 1A1 enzyme is responsible for the conjugation and detoxification of SN-38, the active form of irinotecan. Irinotecan is an anticancer drug used in the treatment of colorectal and lung cancers. Mutations in the UGT1A1 gene cause altered (e.g., reduced or increased) enzymatic activity. Reduced activity can lead to toxicity due to the excessive accumulation of SN-38, resulting in diarrhea and leukopenia. A TA insertion in the highly repetitive TATA-box of the gene promoter is the most common type of UGT1A1 variant. The wild-type allele is referred to as (TA)6. The (TA)7 allele, UGT1A1*28, leads to decreased metabolism of irinotecan and is also associated with Gilbert's Syndrome, a benign form of unconjugated bilirubinemia.

The embodiments described below provide assays designed to distinguish between wild-type (TA)₆ and insertion mutation (TA)₇ sequences and to detect the deletion of a TA, (TA)₅, and the insertion of two TA repeats, (TA)₈. The designs provided here can be adapted to the detection of any of these variants.

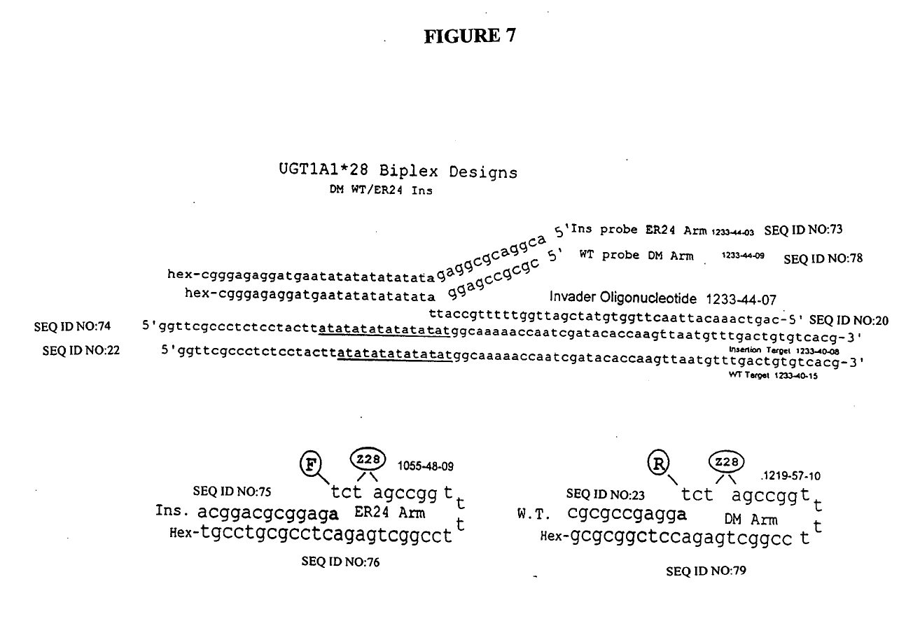

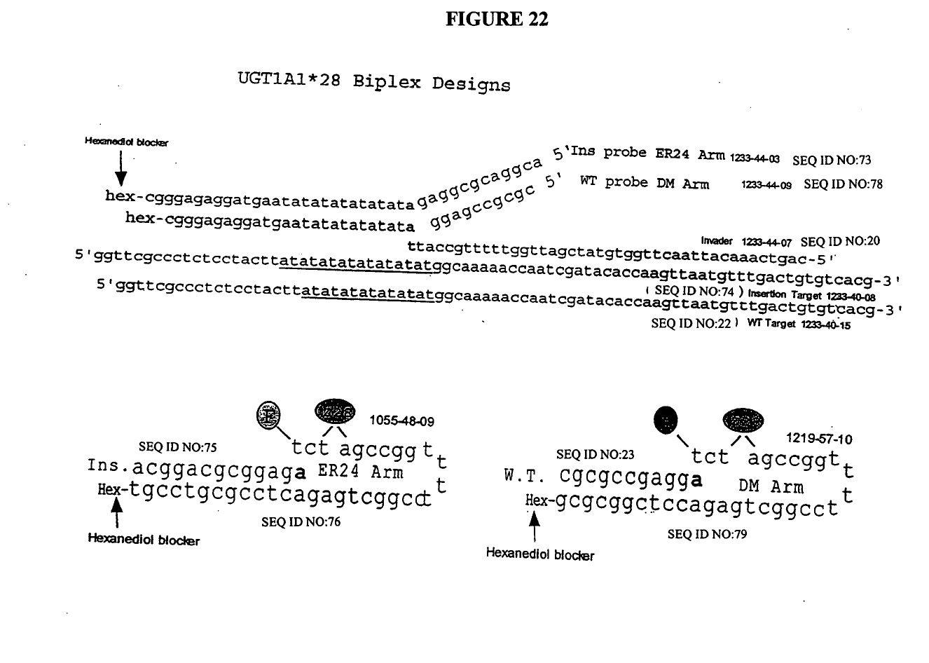

In the preferred embodiments described herein, the probe set targets a T within the TA repeat region of the antisense strand, such that most of the (TA)₆ and (TA)₇ differentiation comes from the analyte-specific region of the probes. Embodiments of this design are shown in FIGS. 6, 7, 10, 12, 13, and 22. In the embodiment shown in FIG. 6, the wild-type probe uses the arm termed “ER38” and is reported by a FRET cassette having the REDMOND RED dye (“Red dye,” Synthetic Genetics, San Diego, Calif.). The insertion probe shown in this embodiment uses the “ER24” arm and is reported by a FRET cassette having the Fam (fluorescein) dye. In an alternative embodiment diagrammed in FIGS. 7 and 22, the WT probe uses the “DM” arm. In preferred embodiments, the probes and the FRET cassettes are blocked on the 3′ ends with hexanediol. In some embodiments, the detection assays are designed to detect (TA)5 and (TA)8. Embodiments of this type of design are shown in 10, 11, and 14.

By way of example, and not intending to limit the procedures of the present invention to any particular configuration or combination of components, the following section describes certain embodiments of a procedure for practicing the present invention:

### EXAMPLE 1

Reaction set-up:

Place 10 ul of sample or control in reaction well.

Overlay with 20 ul Mineral Oil.

Heat to 95 C for 5 minutes to denature.

Cool to 63 C for Reaction Mix addition.

Add 10 ul INVADER Reaction Mix (see below) to each well and mix (e.g., by pipetting).

Incubate at 63 C for 4 hours.

Cool to 4 C to await fluorescence reading.

Warm to room temperature.

Scan in fluorescence plate reader.

INVADER Reaction Mix (Per Reaction):

5 ul DNA Reaction Buffer 1 (14% PEG, 10 mM MOPS pH 7.5, 56 mM MgCl2, 0.02% ProClin 300)

1 ul 1 uM Invader Oligo (in Te)

1 ul 10 uM each WT and Mut Probes (in Te)

1 ul 5 uM Fam FRET (in Te)

1 ul 5 uM Red FRET (in Te)

1 ul 40 ng/ul Cleavase X (in Cleavase Dilution Buffer)

Final reaction concentrations:

3.5% PEG

10 mM MOPs

1.0 pmol INVADER oligonucleotide

10 pmol each primary probe

5 pmol each FRET

40 ng Cleavase X (Third Wave Technologies, Madison, Wis.)

14 mM MgCl2

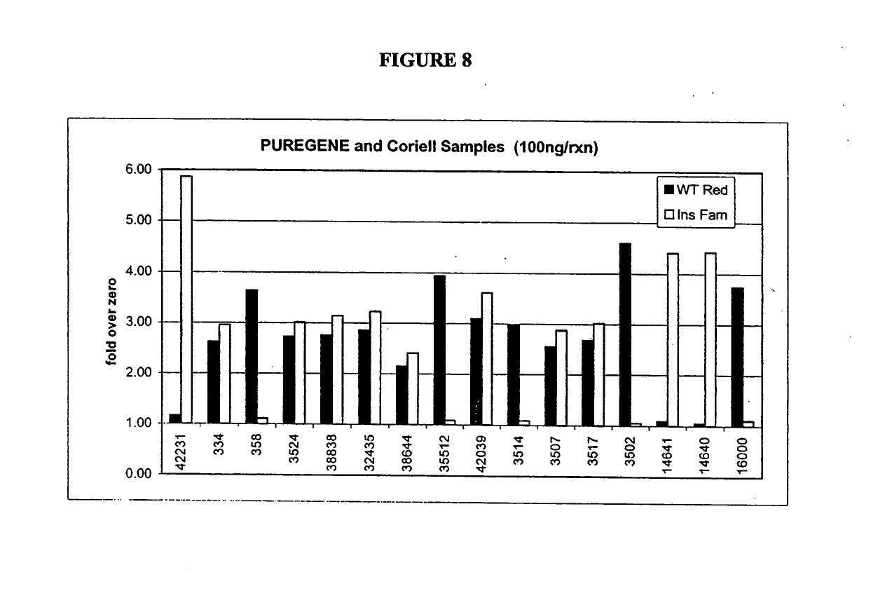

The results of tests run under these conditions are shown in FIG. 8. DNA samples 14641, 14640 and 1600 were purchased from Coreill Institute for Medical Research (Camden, N.J.). The remaining DNA samples were prepared in house using the Gentra PureGene DNA extraction method.

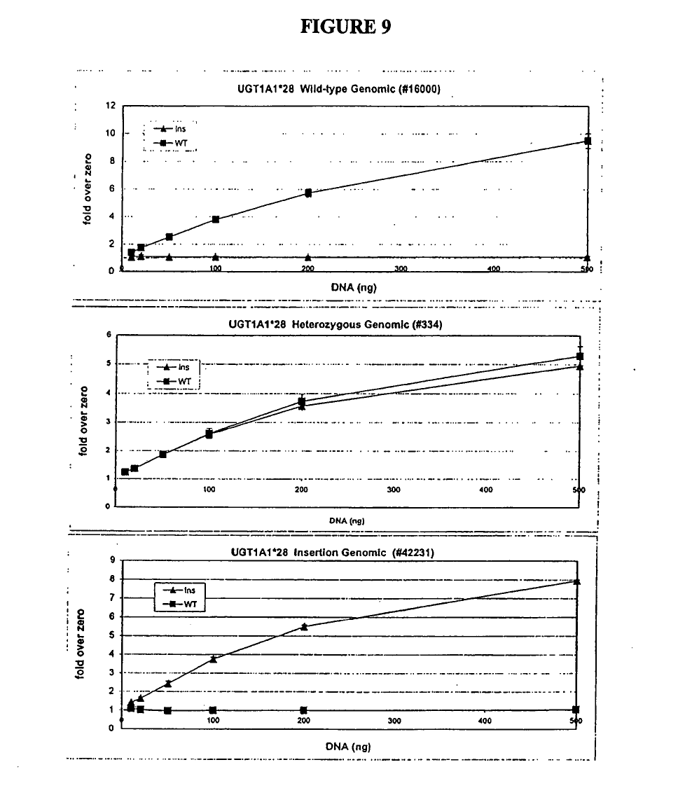

To assess the sensitivity of the assay, DNA samples were tested at concentrations of 10 to 500 ng, with the results diagrammed in FIG. 9. The LOD for each sample was determined by t-test vs. 0, Ratio, and FOZ. The LOD for the wild-type sample was 10 ng by t-test and by Ratio, but 20 ng by FOZ. The highest level of cross-reactivity was 1.11 FOZ. This small amount of cross reactivity did not interfere with the genotype call by the INVADER assay. The heterozygous sample had a LOD of 10 ng by t-test and by Ratio, and 50 ng by FOZ. The Het Ratio increased slightly from 0.95 to 1.09 as the amount of DNA increased. There was no cross-reactivity with the wild-type probe on the insertion target. The LOD for the insertion sample by t-test was 10 ng, by FOZ was 50 ng, and by Ratio was 20 ng. There was no cross-reactivity with the wild-type probe one the insertion target.

These data demonstrate the application of the INVADER assay to the detection of polymorphisms comprising short tandem repeat sequences.

### EXAMPLE 2

TA5 and TA8 INVADER Assays

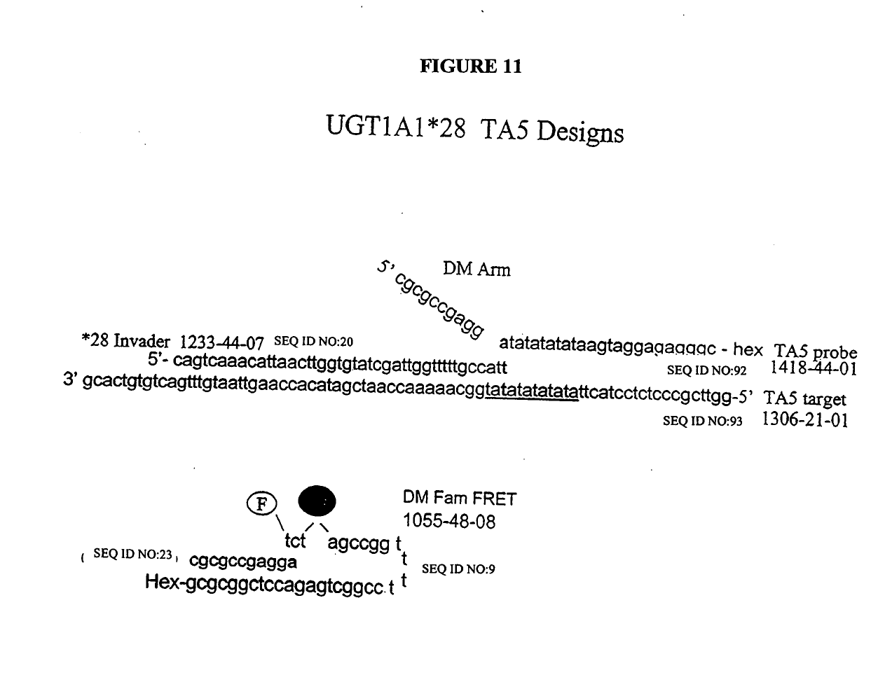

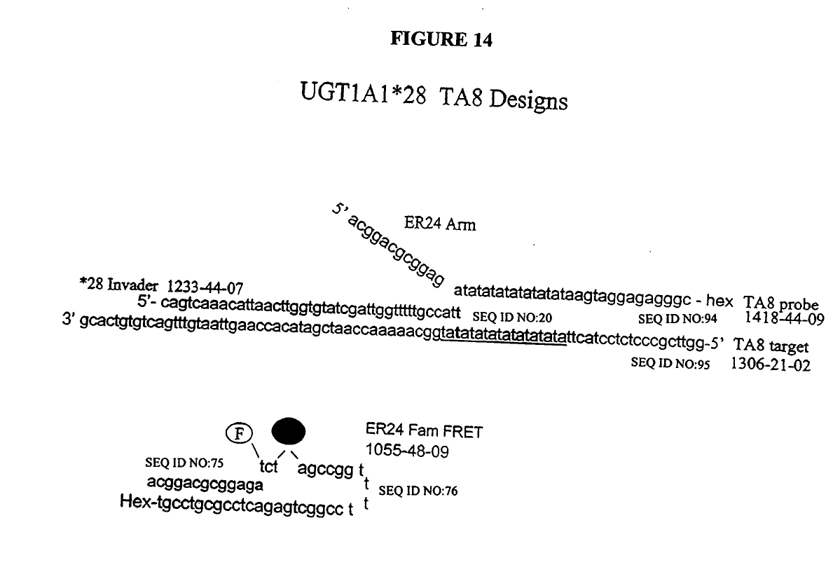

The example describes performing TA5 and TA8 UGT1A1 detection with the INVADER assay. The INVADER assay design for TA5 in this example is shown in FIG. 11 and the INVADER assay design for TA8 in this example is shown in FIG. 14. The TA5 and TA8 monoplex assays were run across the same set of genomic samples and synthetic targets. In both cases, the probes reported to Fam dye. The following assay conditions were employed:

ASR 10:10 Reaction Format:

Place 10 ul of sample or control in reaction well.

Overlay with 20 ul Mineral Oil.

Heat to 95 C for 5 minutes to denature.

Cool to 63 C for Reaction Mix addition.

Add 10 ul Invader Reaction Mix (see below) to each well; mix by pipetting.

Incubate at 63 C for 4 hours.

Cool to 4 C to await fluorescence reading.

Warm to room temperature.

Scan in fluorescence plate reader

Invader Reaction Mix (Per Reaction):

5 ul DNA Reaction Buffer 1 (14% PEG, 40 mM MOPS pH 7.5, 56 mM MgCl2, 0.02% ProClin 300)

1 ul 1 uM Invader Oligo (in Te)

1 ul 10 uM UGT1A1*28 probe (in Te)

0.5 ul 10 uM Fam FRET(in Te)

1 ul 40 ng/ul Cleavase X (in Cleavase Dilution Buffer)

1.5 ul water

Final Reactions Concentrations:

3.5% PEG

10 mM MOPs

1.0 pmol Invader

10 pmol primary probe

5 pmol FRET

40 ng Cleavase X

14 mM MgCl2

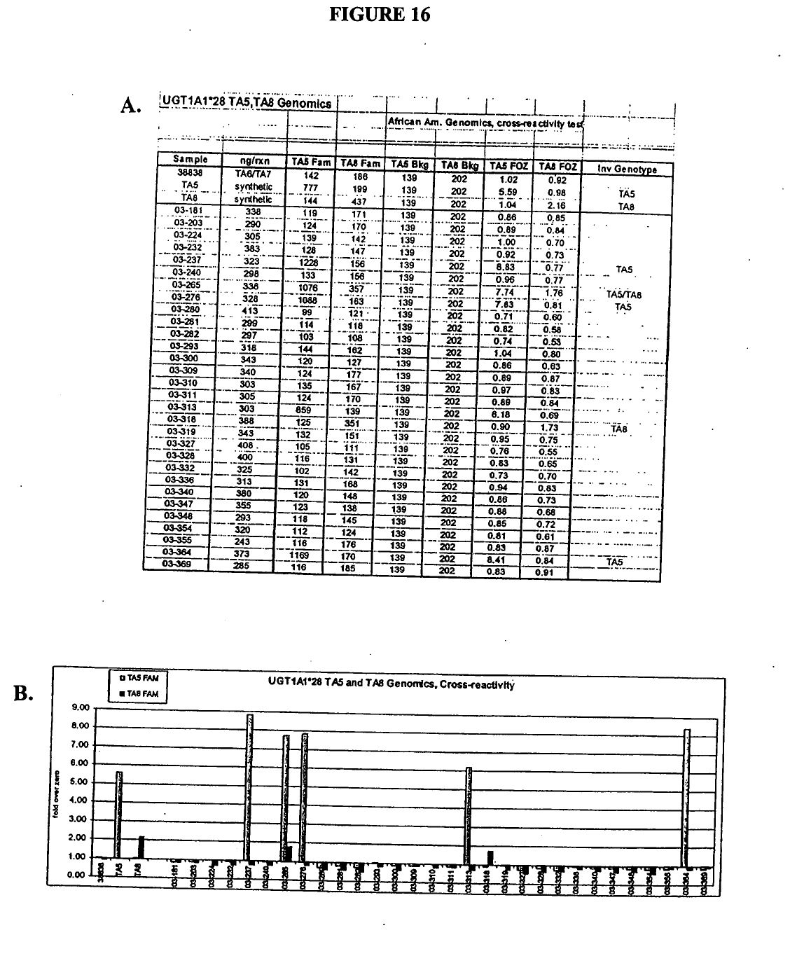

The first three samples were run to test for cross-reactivity. Both the TA5 and TA8 assays were run with a TA6/TA7 genomic Het sample, 38838. The TA5 assay was tested on the TA8 synthetic target, and the TA8 assay was tested on the TA5 synthetic target. The TA5 probe only produced signal with the TA5 target. The TA8 probe only produced signal with the TA8 target. There was no cross-reactivity with the genomic sample 38838. This indicates that the TA5 probe does not cross-react with the TA6, TA7, TA8 sequences, and the TA8 probes does not cross-react with the TA5, TA6, TA7 sequences (See, FIG. 16A).

The remaining genomic DNAs were screened for the TA5 and TA8 alleles. Samples 03-237, 03-265, 03-276, 03-313, 03-364 showed signal with the TA5 assay (See, FIG. 16B). Samples 03-265 and 03-318 showed signal with the TA8 assay (See, FIG. 16B).

The six genomic samples that showed signal in the TA5 and TA8 assays were run in the TA5 and TA8 assays were then run in the TA6/TA7 biplex assay. The UGT1A1*28 INVADER genotypes for these six samples is shown below.

| Sample | UGT1A1*28 Genotype |
|---|---|
| 03-237 | TA5/TA6 |
| 03-265 | TA5/TA8 |
| 03-276 | TA5/TA7 |
| 03-313 | TA5/TA7 |
| 03-318 | TA6/TA8 |
| 03-364 | TA5/TA6 |

The set up for TA6/TA7 biplex assay was as follows.
ASR 10:10 Reaction Format:

Place 10 ul of sample or control in reaction well.

Overlay with 20 ul Mineral Oil.

Heat to 95 C for 5 minutes to denature.

Cool to 63 C for Reaction Mix addition.

Add 10 ul Invader Reaction Mix (see below) to each well;

mix by pipetting.

Incubate at 63 C for 4 hours.

Cool to 4 C to await fluorescence reading.

Warm to room temperature.

Scan in fluorescence plate reader.

Invader Reaction Mix (Per Reaction):

5 ul DNA Reaction Buffer 1 (14% PEG, 40 mM MOPS pH 7.5, 56 mM MgCl2, 0.02% ProClin 300)

1 ul 1 uM Invader Oligo (in Te)

1 ul 10 uM each WT and Mut Probes (in Te)

1 ul 5 uM Fam FRET (in Te)

1 ul 5 uM Red FRET (in Te)

1 ul 40 ng/ul Cleavase X (in Cleavase Dilution Buffer)

Final Reactions Concentrations:

3.5% PEG

10 mM MOPs

1.0 pmol Invader

10 pmol each primary probe

5 pmol each FRET

40 ng Cleavase X

14 mM MgCl2

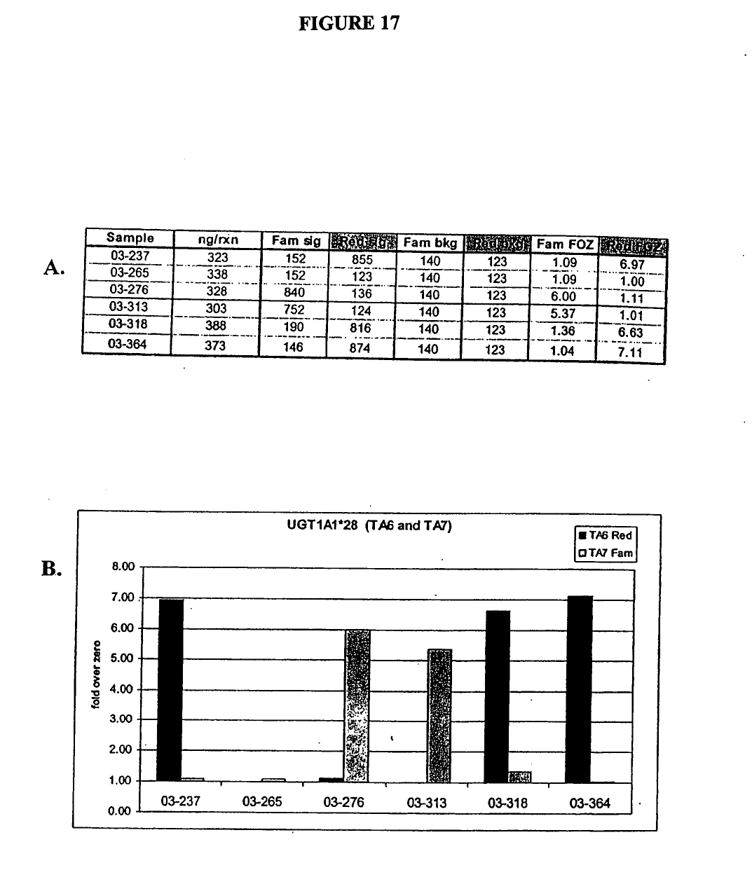

The five samples that were positive for either TA5 or TA8 (above) were also positive for either the TA6 or TA7 allele. Sample 03-265 was positive for both TA5 and TA8. In the TA6/TA7 assay, this sample resulted in no signal (See, FIG. 17A-B). This indicates that the TA6 and TA7 probes are not cross-reactive with the TA5 or TA8 sequences.

### UGT Example 3

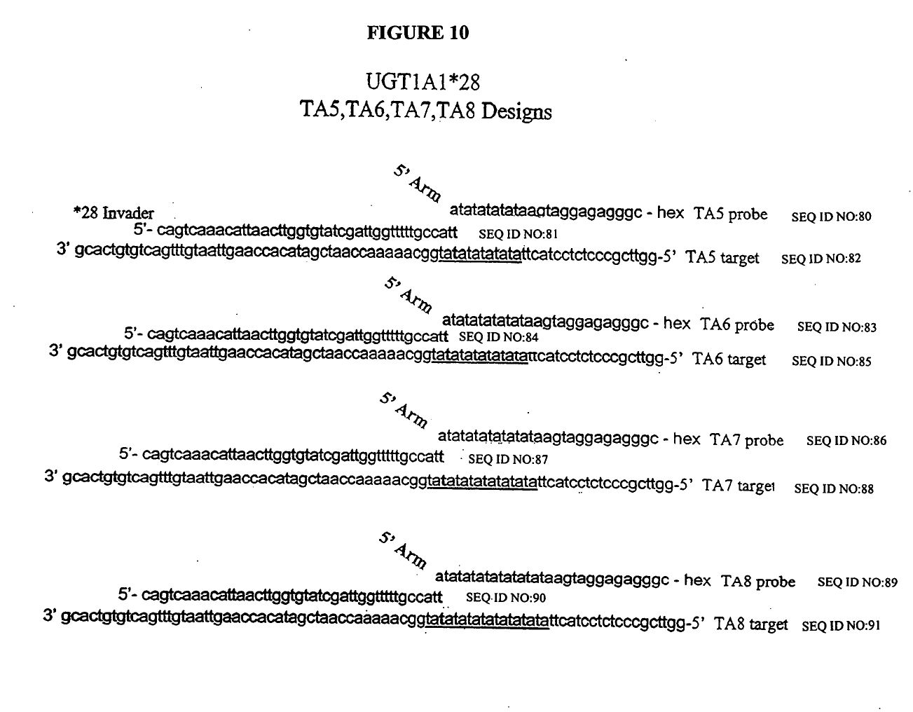

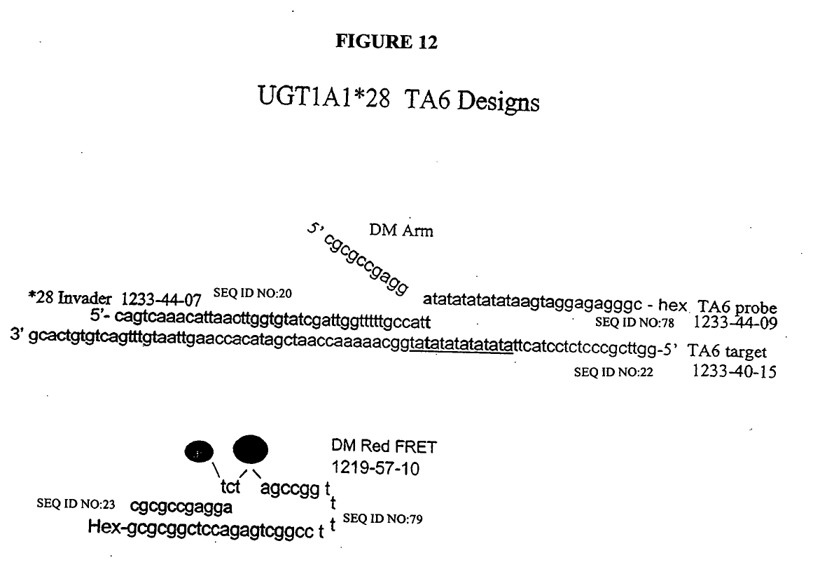

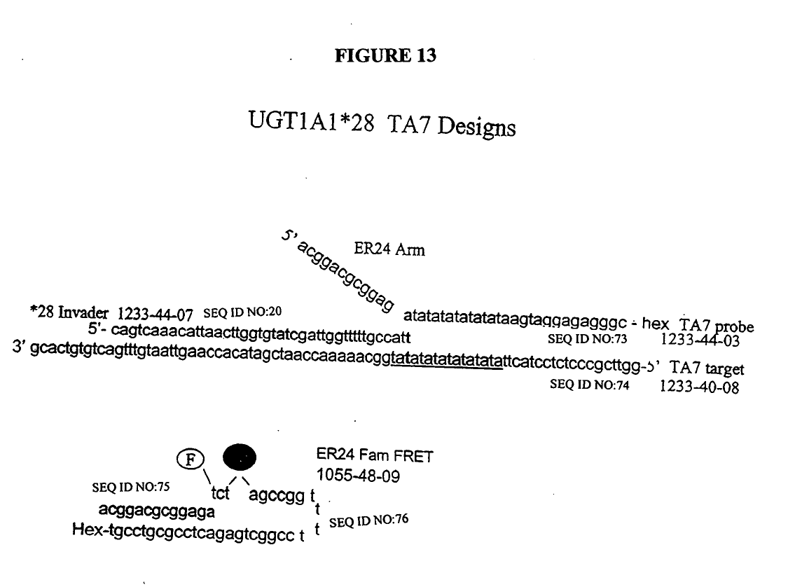

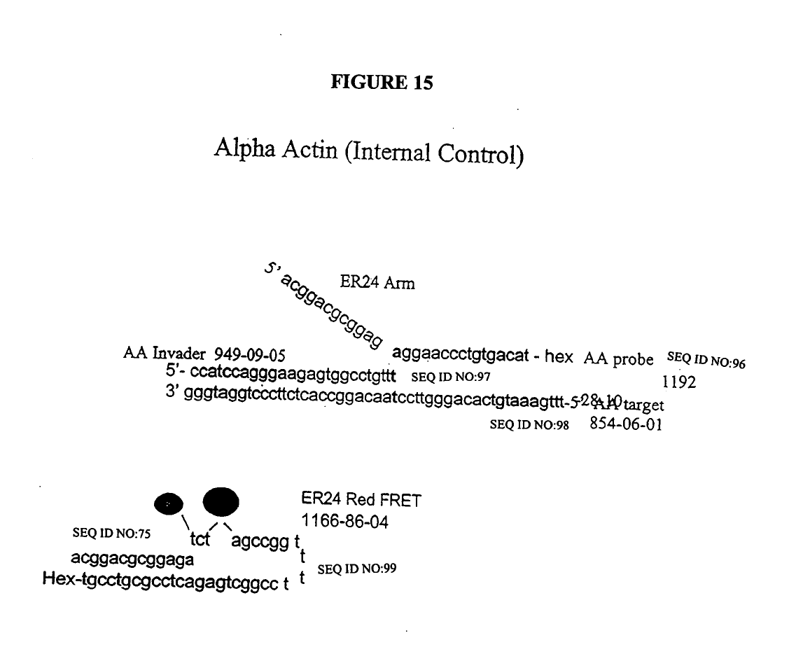

### UGT1A1*28 Biplexed with Internal Control This example describes one embodiment for a UGT1A1*28 Assay with an Internal Control. The assay may be designed as a 4 well assay in which each *28 probe (TA5, TA6, TA7, and TA8) are biplexed with an internal control. This assay may employ the INVADER assay for one or more of the *28 probes. FIG. 10 shows useful INVADER assay configurations for TA5, TA6, TA7 and TA8, that may be biplexed with the Alpha Actin internal control shown in FIG. 15. Other useful INVADER configurations that may be employed are shown in FIG. 11 (TA5), FIG. 12 (TA6), FIG. 13 (TA7), and FIG. 14 (TA8), which may be biplexed with the internal control shown in FIG. 15.

Assay set up conditions that may be employed to set up this 4 well assay are as follows.

ASR 10:10 Reaction Format:

Place 10 ul of sample or control in reaction well.
Overlay with 20 ul Mineral Oil.
Heat to 95 C for 5 minutes to denature.
Cool to 63 C for Reaction Mix addition.
Add 10 ul Invader Reaction Mix (see below) to each well;
mix by pipetting.
Incubate at 63 C for 4 hours.
Cool to 4 C to await fluorescence reading.
Warm to room temperature.
Scan in fluorescence plate reader.

Invader Reaction Mix (Per Reaction):
5 ul DNA Reaction Buffer 1 (14% PEG, 40 mM MOPS pH 7.5, 56 mM MgCl2, 0.02% ProClin 300)
0.5 ul 2 uM *28 Invader Oligo (in Te)
0.5 ul 2 uM IC Invader Oligo (in Te)
0.5 ul 20 uM *28 Probe (in Te)
0.5 ul 20 uM IC Probe (in Te)
1 ul 5 uM Fam FRET (in Te)
1 ul 5 uM Red FRET (in Te)
1 ul 40 ng/ul Cleavase X (in Cleavase Dilution Buffer)
   Final reactions concentrations:
3.5% PEG
10 mM MOPs
1.0 pmol each Invader
10 pmol each primary probe
5 pmol each FRET
40 ng Cleavase X
14 mM MgCl2

As the UGT examples above show, the INVADER assay may be configured to detect repeat sequences. The INVADER assay may be configured to detect repeat sequences in other target nucleic acid sequence (e.g. other drug metabolizing genes) that contain repeat sequences. Preferably, the INVADER assay is employed to detect repeat sequences (e.g. in genomic DNA) that are determined to be associated with a particular condition (e.g. predisposition to disease, altered drug metabolism, etc.). For example, INVADER assays may be configured to detect tandemly repetitive sequences, such as satellites, minisatellites, and microsatellites (See, e.g. Bennet, J. Clin. Pathol: Mol. Pathol., 2000; 53:177-183, herein incorporated by reference in its entirity). INVADER assays may also be configured to detect interspersed repetitive DNA sequences such as SINE (e.g. Alu repeat) and LINES. In certain embodiments, the INVADER assays are configured to detect short tandem repeats (STRs) for applications such as forensics and paternity testing (e.g. Tracey, Croatian Medical Journal, 42(3):233-238, 2001, herein incorporated by reference, see also the Marshfield Clinic web site for lists of target repeat sequences for which INVADER assay may be configured to detect). In other embodiments, INVADER assay are configured to detect repeat sequences in plants (e.g. crop plants).

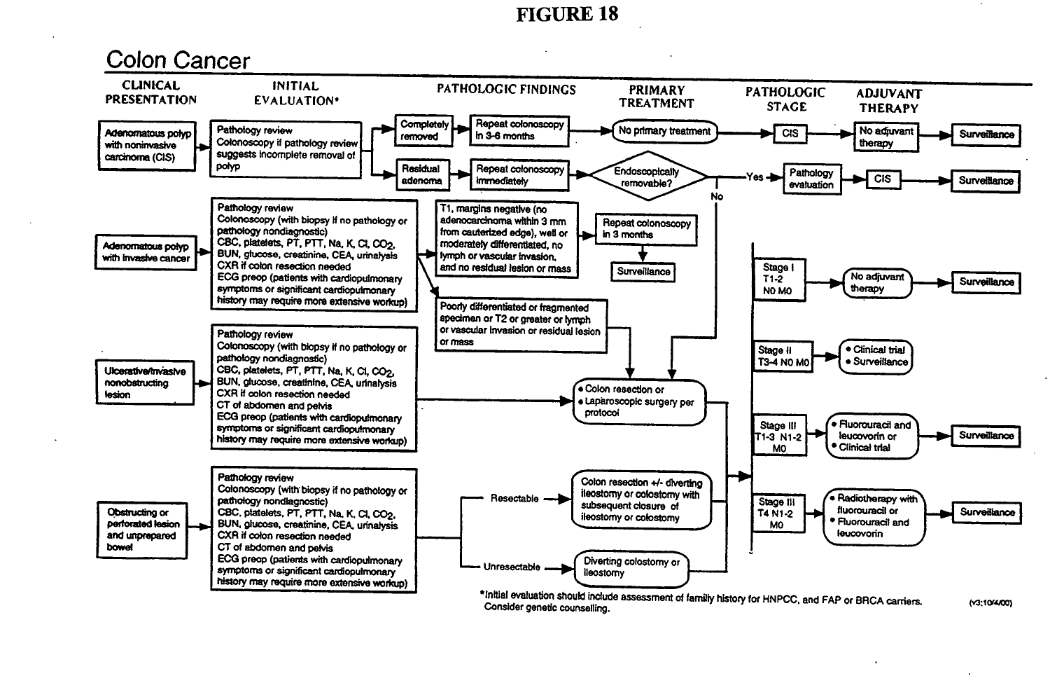

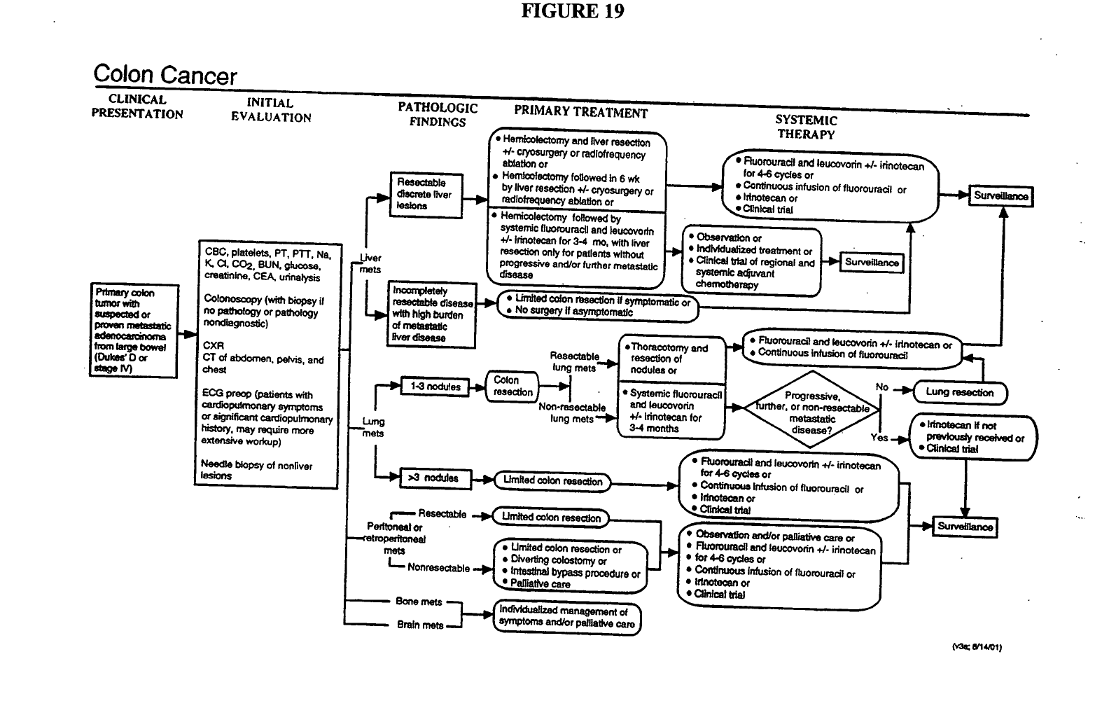

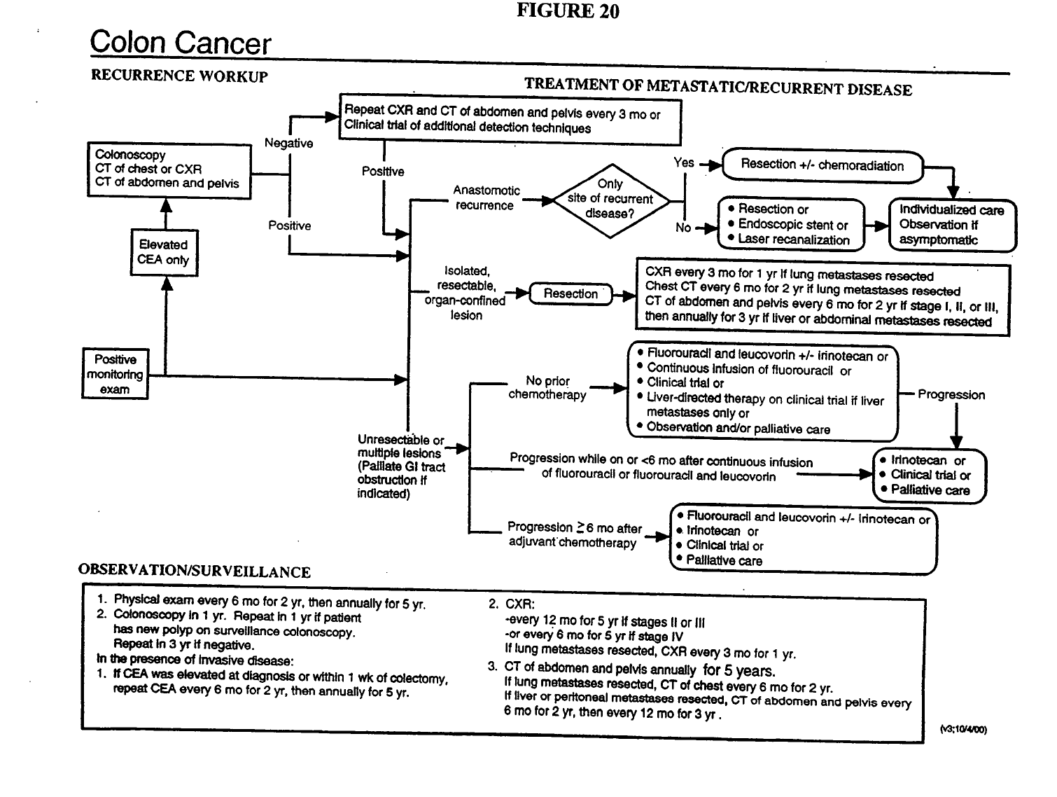

The UGT repeat detection assays of the present invention may also be used in combination with drug therapy (e.g. irinotecan) and additional treatment and/or diagnostic procedures. For example, this combination of UGT detection assays, drug therapy and additional treatment/diagnostic protocols may be applied to the management of colon cancer or lung cancer. For example, FIGS. 18, 19 and 20 show various colon cancer practice guidelines created by the National Comprehensive Cancer Network and modified by the University of Texas M.D. Anderson Cancer Center (See additional management protocols in Adenis et al., Elec. J. of Oncology, 2001, 1, 83-89, herein incorporated by reference). These colon cancer protocols often call for the administration of irinotecan. As such, employing the UGT detection assays of the present invention at one or more places in these colon cancer management flow charts is a useful step in successful patient care.

The following applications are incorporated herein by reference in their entireties:

U.S. Provisional Application 60/353,166, filed Jan. 31, 2002;

U.S. Provisional Application 60/353,167, filed Jan. 31, 2002;

U.S. Provisional Application 60/353,165, filed Jan. 31, 2002;

U.S. Provisional Application 60/366,984, filed Mar. 22, 2002;

U.S. Provisional Application 60/424,578, filed Nov. 7, 2002;

U.S. application Ser. No. 10/035,833, filed Dec. 27, 2001;

U.S. Provisional Application 60/371, 819, filed Apr. 11, 2002;

U.S. Provisional Application 60/352,940, filed Jan. 30, 2002; and

U.S. Provisional Application 60/356,326, filed Feb. 13, 2002;

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

1. A composition comprising a detection assay configured for detecting at least one polymorphism in UGT1A1, wherein said detection assay comprises a primary probe, an INVADER oligonucleotide, a structure specific enzyme, and a FRET cassette.

2. The composition of claim 1, wherein said at least one polymorphism in UGT1A1 is selected from UGT1A1 *6, *27, *28, and the polymorphisms shown in FIG. 4.

3. The composition of claim 1, wherein said primary probe comprises a 5′ flap.

4. The composition of claim 1, wherein said detection assay comprises at least one oligonucleotide comprising a sequence shown in FIG. 1, 2, 3, 5, 6, 7, 10, 11, 12, 13, 14, 15, 21, or 22.

5. The composition of claim 1, wherein said detection assay comprises at least one oligonucleotide consisting of a sequence shown in FIG. 1, 2, 3, 5, 6, 7, 10, 11, 12, 13, 14, 15, 21, or 22.

6. The composition of claim 1, wherein said detection assay comprises at least one oligonucleotide consisting of a first portion having a sequence selected from the group consisting of SEQ ID NOS: 80, 83, 86 and 89, and a second portion consisting of a 5′ arm.

7. The composition of claim 1, wherein said composition further comprises a second detection assay, wherein said second detection assay is configured to detect a control nucleic acid.

8. The composition of claim 7, wherein said control nucleic acid is a wild type allele of UGT1A1 with respect to the locus of said at least one polymorphism.

9. The composition of claim 7, wherein said control nucleic acid is from a housekeeping gene.

10. The method of claim 9, wherein said housekeeping gene is α-actin.

11. A method comprising;
a) providing:
i) a composition comprising a detection assay configured for detecting a UGT1A1 polymorphism, and
ii) a sample from a subject; and
b) testing said sample with said composition in order to determine if said subject has said UGT1A1 polymorphism.
wherein said non-amplified oligonucleotide detection assay comprises a primary probe, an INVADER oligonucleotide, a structure specific enzyme, and a FRET cassette.

12. The method of claim 11, wherein said UGT1A1 polymorphism is selected from UGT1A1 *6, *27, *28, and the polymorphisms shown in FIG. 4.

13. The method of claim 11, wherein said primary probe comprises a 5′ flap.

14. The method of claim 11, wherein said detection assay comprises at least one sequence shown in FIG. 1, 2, 3, 5, 6, 7, 10, 11, 12, 13, 14, 15, 21, or 22.

15. The method of claim 11, wherein said detection assay comprises an oligonucleotide consisting of a first portion having a sequence selected from the group consisting of SEQ ID NOS: 80, 83, 86 and 89, and a second portion consisting of a 5′ arm.

16. The method of claim 11, comprising further providing a second detection assay, wherein said second detection assay is configured to detect a control nucleic acid.

17. The method of claim 16, wherein said control nucleic acid is a wild type allele of UGT1A1 with respect to the locus of said at least one polymorphism.

18. The method of claim 16, wherein said control nucleic acid is from a housekeeping gene.

19. The method of claim 16, wherein said housekeeping gene is α-actin.


SEQUENCE LISTING (102 sequences)

SEQ ID NO: 1 - DNA, 28 bp, Artificial Sequence
  Synthetic
cgcgccgaggtgtctctgatgtacaacg

SEQ ID NO: 2 - DNA, 29 bp, Artificial Sequence
  Synthetic
tccgcgcgtcccgtctctgatgtacaacg

SEQ ID NO: 3 - DNA, 35 bp, Artificial Sequence
  Synthetic
ggcacagggtacgtcttcaaggtgtaaaatgctca

SEQ ID NO: 4 - DNA, 62 bp, Artificial Sequence
  Synthetic
cgcctcgttgtacatcagagacagagcattttacaccttgaagacgtaccctgtgccatttt

SEQ ID NO: 5 - DNA, 62 bp, Artificial Sequence
  Synthetic
cgcctcgttgtacatcagagacggagcattttacaccttgaagacgtaccctgtgccatttt

SEQ ID NO: 6 - DNA, 12 bp, Artificial Sequence
  Synthetic
tccgcgcgtccc

SEQ ID NO: 7 - DNA, 34 bp, Artificial Sequence
  Synthetic
tcttcggccttttggccgagagaggacgcgcgga

SEQ ID NO: 8 - DNA, 11 bp, Artificial Sequence
  Synthetic
cgcgccgaggt

SEQ ID NO: 9 - DNA, 33 bp, Artificial Sequence
  Synthetic
tctagccggttttccggctgagacctcggcgcg

SEQ ID NO: 10 - DNA, 27 bp, Artificial Sequence
  Synthetic
tccgcgcgtcccgtatgcaacccttgc

SEQ ID NO: 11 - DNA, 28 bp, Artificial Sequence
  Synthetic
aggccacggacgagtatgcaacccttgc

SEQ ID NO: 12 - DNA, 40 bp, Artificial Sequence
  Synthetic
cttttcacagaactttctgtgcgacgtggttttattccct

SEQ ID NO: 13 - DNA, 63 bp, Artificial Sequence
  Synthetic
tgaggcaagggttgcatacggggaataaaccacgtcgcacagaaagttctgtgaaaaggcttt

SEQ ID NO: 14 - DNA, 63 bp, Artificial Sequence
  Synthetic
tgaggcaagggttgcatactgggaataaaccacgtcgcacagaaagttctgtgaaaaggcttt

SEQ ID NO: 15 - DNA, 34 bp, Artificial Sequence
  Synthetic
tctagccggttttccggctgagaggacgcgcgga

SEQ ID NO: 16 - DNA, 13 bp, Artificial Sequence
  Synthetic
aggccacggacga

SEQ ID NO: 17 - DNA, 35 bp, Artificial Sequence
  Synthetic
tcttcggccttttggccgagagacgtccgtggcct

SEQ ID NO: 18 - DNA, 38 bp, Artificial Sequence
  Synthetic
cgcgccgaggatatatatatatataagtaggagagggc

SEQ ID NO: 19 - DNA, 38 bp, Artificial Sequence
  Synthetic
aggccacggacgatatatatatataagtaggagagggc

SEQ ID NO: 20 - DNA, 42 bp, Artificial Sequence
  Synthetic
cagtcaaacattaacttggtgtatcgattggtttttgccatt

SEQ ID NO: 21 - DNA, 81 bp, Artificial Sequence
  Synthetic
ggttcgccctctcctacttatatatatatatatatggcaaaaaccaatcgatacaccaagttaatgtttgactgtgtcacg

SEQ ID NO: 22 - DNA, 79 bp, Artificial Sequence
  Synthetic
ggttcgccctctcctacttatatatatatatatggcaaaaaccaatcgatacaccaagttaatgtttgactgtgtcacg

SEQ ID NO: 23 - DNA, 11 bp, Artificial Sequence
  Synthetic
cgcgccgagga

SEQ ID NO: 24 - DNA, 41 bp, Artificial Sequence
  Synthetic
  misc_feature (21)..(21): n is a or c.
tctttcccttttgacttcaantcagtcatcagaatttcccc

SEQ ID NO: 25 - DNA, 41 bp, Artificial Sequence
  Synthetic
  misc_feature (21)..(21): n is g or a.
cctcgttgtacatcagagacngagcattttacaccttgaag

SEQ ID NO: 26 - DNA, 41 bp, Artificial Sequence
  Synthetic
  misc_feature (21)..(21): n is t or g.
gcatttgggaagggaaaatcnaattaaaagcctaaactaaa

SEQ ID NO: 27 - DNA, 41 bp, Artificial Sequence
  Synthetic
  misc_feature (21)..(21): n is g or t.
agactcggccttttccagatnagcttcagtgtaagagtggg

SEQ ID NO: 28 - DNA, 41 bp, Artificial Sequence
  Synthetic
  misc_feature (21)..(21): n is c or t.
ttaagtaagccatttaccaangctcagaagaaagaacttga

SEQ ID NO: 29 - DNA, 41 bp, Artificial Sequence
  Synthetic
  misc_feature (21)..(21): n is t or c.
tcttgctacaaaccaaaaaangcagcatggtggtggggagg

SEQ ID NO: 30 - DNA, 41 bp, Artificial Sequence
  Synthetic
  misc_feature (21)..(21): n is t or c.
cagacagtaagaagattctanaccatggcctcatatctatt

SEQ ID NO: 31 - DNA, 41 bp, Artificial Sequence
  Synthetic
  misc_feature (21)..(21): n is c or t.
agatttaaaactccaatttanataaaaagttgccataatag

SEQ ID NO: 32 - DNA, 41 bp, Artificial Sequence
  Synthetic
  misc_feature (21)..(21): n is c or t.
tatagaggttcacacacacangccttcattgcgtgtgcatg

SEQ ID NO: 33 - DNA, 21 bp, Artificial Sequence
  Synthetic
tatagaggttcacacacacaa

SEQ ID NO: 34 - DNA, 29 bp, Artificial Sequence
  Synthetic
atgacgtggcagaccgccttcattgcgtg

SEQ ID NO: 35 - DNA, 25 bp, Artificial Sequence
  Synthetic
cgcgccgaggtgccttcattgcgtg

SEQ ID NO: 36 - DNA, 39 bp, Artificial Sequence
  Synthetic
tgcacacgcaatgaaggcgtgtgtgtgtgaacctctata

SEQ ID NO: 37 - DNA, 39 bp, Artificial Sequence
  Synthetic
tgcacacgcaatgaaggcatgtgtgtgtgaacctctata

SEQ ID NO: 38 - DNA, 21 bp, Artificial Sequence
  Synthetic
tctttcccttttgacttcaat

SEQ ID NO: 39 - DNA, 30 bp, Artificial Sequence
  Synthetic
cgcgccgaggatcagtcatcagaatttccc

SEQ ID NO: 40 - DNA, 34 bp, Artificial Sequence
  Synthetic
atgacgtggcagacctcagtcatcagaatttccc

SEQ ID NO: 41 - DNA, 41 bp, Artificial Sequence
  Synthetic
ggggaaattctgatgactgatttgaagtcaaaagggaaaga

SEQ ID NO: 42 - DNA, 41 bp, Artificial Sequence
  Synthetic
ggggaaattctgatgactgagttgaagtcaaaagggaaaga

SEQ ID NO: 43 - DNA, 21 bp, Artificial Sequence
  Synthetic
tttagtttaggcttttaattt

SEQ ID NO: 44 - DNA, 29 bp, Artificial Sequence
  Synthetic
cgcgccgaggagattttcccttcccaaat

SEQ ID NO: 45 - DNA, 32 bp, Artificial Sequence
  Synthetic
atgacgtggcagaccgattttcccttcccaaa

SEQ ID NO: 46 - DNA, 41 bp, Artificial Sequence
  Synthetic
gcatttgggaagggaaaatctaattaaaagcctaaactaaa

SEQ ID NO: 47 - DNA, 41 bp, Artificial Sequence
  Synthetic
gcatttgggaagggaaaatcgaattaaaagcctaaactaaa

SEQ ID NO: 48 - DNA, 21 bp, Artificial Sequence
  Synthetic
cccactcttacactgaagctt

SEQ ID NO: 49 - DNA, 30 bp, Artificial Sequence
  Synthetic
atgacgtggcagaccatctggaaaaggccg

SEQ ID NO: 50 - DNA, 26 bp, Artificial Sequence
  Synthetic
cgcgccgaggaatctggaaaaggccg

SEQ ID NO: 51 - DNA, 40 bp, Artificial Sequence
  Synthetic
gactcggccttttccagatgagcttcagtgtaagagtggg

SEQ ID NO: 52 - DNA, 40 bp, Artificial Sequence
  Synthetic
gactcggccttttccagattagcttcagtgtaagagtggg

SEQ ID NO: 53 - DNA, 21 bp, Artificial Sequence
  Synthetic
ttaagtaagccatttaccaaa

SEQ ID NO: 54 - DNA, 33 bp, Artificial Sequence
  Synthetic
atgacgtggcagaccgctcagaagaaagaactt

SEQ ID NO: 55 - DNA, 30 bp, Artificial Sequence
  Synthetic
cgcgccgaggtgctcagaagaaagaacttg

SEQ ID NO: 56 - DNA, 41 bp, Artificial Sequence
  Synthetic
tcaagttctttcttctgagcgttggtaaatggcttacttaa

SEQ ID NO: 57 - DNA, 41 bp, Artificial Sequence
  Synthetic
tcaagttctttcttctgagcattggtaaatggcttacttaa

SEQ ID NO: 58 - DNA, 21 bp, Artificial Sequence
  Synthetic
cctccccaccaccatgctgct

SEQ ID NO: 59 - DNA, 31 bp, Artificial Sequence
  Synthetic
cgcgccgaggattttttggtttgtagcaaga

SEQ ID NO: 60 - DNA, 35 bp, Artificial Sequence
  Synthetic
atgacgtggcagacgttttttggtttgtagcaaga

SEQ ID NO: 61 - DNA, 41 bp, Artificial Sequence
  Synthetic
tcttgctacaaaccaaaaaatgcagcatggtggtggggagg

SEQ ID NO: 62 - DNA, 41 bp, Artificial Sequence
  Synthetic
tcttgctacaaaccaaaaaacgcagcatggtggtggggagg

SEQ ID NO: 63 - DNA, 21 bp, Artificial Sequence
  Synthetic
cagacagtaagaagattctaa

SEQ ID NO: 64 - DNA, 30 bp, Artificial Sequence
  Synthetic
cgcgccgaggtaccatggcctcatatctat

SEQ ID NO: 65 - DNA, 31 bp, Artificial Sequence
  Synthetic
atgacgtggcagaccaccatggcctcatatc

SEQ ID NO: 66 - DNA, 41 bp, Artificial Sequence
  Synthetic
aatagatatgaggccatggtatagaatcttcttactgtctg

SEQ ID NO: 67 - DNA, 41 bp, Artificial Sequence
  Synthetic
aatagatatgaggccatggtgtagaatcttcttactgtctg

SEQ ID NO: 68 - DNA, 21 bp, Artificial Sequence
  Synthetic
ctattatggcaactttttatt

SEQ ID NO: 69 - DNA, 35 bp, Artificial Sequence
  Synthetic
atgacgtggcagacgtaaattggagttttaaatct

SEQ ID NO: 70 - DNA, 31 bp, Artificial Sequence
  Synthetic
cgcgccgaggataaattggagttttaaatct

SEQ ID NO: 71 - DNA, 41 bp, Artificial Sequence
  Synthetic
agatttaaaactccaatttacataaaaagttgccataatag

SEQ ID NO: 72 - DNA, 41 bp, Artificial Sequence
  Synthetic
agatttaaaactccaatttatataaaaagttgccataatag

SEQ ID NO: 73 - DNA, 40 bp, Artificial Sequence
  Synthetic
acggacgcggagatatatatatatataagtaggagagggc

SEQ ID NO: 74 - DNA, 81 bp, Artificial Sequence; identical to SEQ ID NO: 21
  Synthetic

SEQ ID NO: 75 - DNA, 13 bp, Artificial Sequence
  Synthetic
acggacgcggaga

SEQ ID NO: 76 - DNA, 35 bp, Artificial Sequence
  Synthetic
tctagccggttttccggctgagactccgcgtccgt

SEQ ID NO: 77 - DNA, 35 bp, Artificial Sequence
  Synthetic
tctagccggttttccggctgagacgtccgtggcct

SEQ ID NO: 78 - DNA, 36 bp, Artificial Sequence
  Synthetic
cgcgccgaggatatatatatataagtaggagagggc

SEQ ID NO: 79 - DNA, 33 bp, Artificial Sequence; identical to SEQ ID NO: 9
  Synthetic

SEQ ID NO: 80 - DNA, 24 bp, Artificial Sequence
  Synthetic
atatatatataagtaggagagggc

SEQ ID NO: 81 - DNA, 42 bp, Artificial Sequence; identical to SEQ ID NO: 20
  Synthetic

SEQ ID NO: 82 - DNA, 77 bp, Artificial Sequence
  Synthetic
ggttcgccctctcctacttatatatatatatggcaaaaaccaatcgatacaccaagttaatgtttgactgtgtcacg

SEQ ID NO: 83 - DNA, 26 bp, Artificial Sequence
  Synthetic
atatatatatataagtaggagagggc

SEQ ID NO: 84 - DNA, 42 bp, Artificial Sequence; identical to SEQ ID NO: 20
  Synthetic

SEQ ID NO: 85 - DNA, 79 bp, Artificial Sequence; identical to SEQ ID NO: 22
  Synthetic

SEQ ID NO: 86 - DNA, 28 bp, Artificial Sequence
  Synthetic
atatatatatatataagtaggagagggc

SEQ ID NO: 87 - DNA, 42 bp, Artificial Sequence; identical to SEQ ID NO: 20
  Synthetic

SEQ ID NO: 88 - DNA, 81 bp, Artificial Sequence; identical to SEQ ID NO: 21
  Synthetic

SEQ ID NO: 89 - DNA, 30 bp, Artificial Sequence
  Synthetic
atatatatatatatataagtaggagagggc

SEQ ID NO: 90 - DNA, 42 bp, Artificial Sequence; identical to SEQ ID NO: 20
  Synthetic

SEQ ID NO: 91 - DNA, 83 bp, Artificial Sequence
  Synthetic
ggttcgccctctcctacttatatatatatatatatatggcaaaaaccaatcgatacaccaagttaatgtttgactgtgtcacg

SEQ ID NO: 92 - DNA, 34 bp, Artificial Sequence
  Synthetic
cgcgccgaggatatatatataagtaggagagggc

SEQ ID NO: 93 - DNA, 77 bp, Artificial Sequence; identical to SEQ ID NO: 82
  Synthetic

SEQ ID NO: 94 - DNA, 42 bp, Artificial Sequence
  Synthetic
acggacgcggagatatatatatatatataagtaggagagggc

SEQ ID NO: 95 - DNA, 83 bp, Artificial Sequence; identical to SEQ ID NO: 91
  Synthetic

SEQ ID NO: 96 - DNA, 28 bp, Artificial Sequence
  Synthetic
acggacgcggagaggaaccctgtgacat

SEQ ID NO: 97 - DNA, 25 bp, Artificial Sequence
  Synthetic
ccatccagggaagagtggcctgttt

SEQ ID NO: 98 - DNA, 47 bp, Artificial Sequence
  Synthetic
tttgaaatgtcacagggttcctaacaggccactcttccctggatggg

SEQ ID NO: 99 - DNA, 35 bp, Artificial Sequence; identical to SEQ ID NO: 76
  Synthetic

SEQ ID NO: 100 - DNA, 26 bp, Artificial Sequence
  Synthetic
cgcgccgaggcgtatgcaacccttgc

SEQ ID NO: 101 - DNA, 11 bp, Artificial Sequence
  Synthetic
cgcgccgaggc

SEQ ID NO: 102 - DNA, 35 bp, Artificial Sequence; identical to SEQ ID NO: 77
  Synthetic